SEQUENCE LISTING (28 sequences)

SEQ ID NO: 1 - protein, 114 aa, synthetic construct
  REGION 1..114: note = mammalian interleukin 15 consensus sequence
  VARIANT 1: note = X= N, S, T or I
  VARIANT 3: note = X= V, H, I, Q or E
  VARIANT 4: note = X= N, Y, F or D
  VARIANT 6: note = X= I or R
  VARIANT 7: note = X= S, N, L, Y, K or R
  VARIANT 10: note = X= K, E, R or Q
  VARIANT 11: note = X= K, T or R
  VARIANT 13: note = X= E, D or Q
  VARIANT 14: note = X= D, H, S or N
  VARIANT 16: note = X= I or T
  VARIANT 17: note = X=Q, R or K
  VARIANT 18: note = X= S or F
  VARIANT 19: note = X= M, I or L
  VARIANT 21: note = X= I, V or M
  VARIANT 23: note = X=A or T
  VARIANT 28: note = X=E or D
  VARIANT 30: note = X= D or G
  VARIANT 31: note = X= V, F, A or I
  VARIANT 34: note = X= S, N or R
  VARIANT 37: note = X= V or I
  VARIANT 39: note = X= A or T
  VARIANT 41: note = X= K, Q or N
  VARIANT 48: note = X= Q, G, R, H or E
  VARIANT 51: note = X= S or L
  VARIANT 52: note = X= L, Q or H
  VARIANT 54: note = X= S, F or Y
  VARIANT 55: note = X= G, K, S, N or R
  VARIANT 56: note = X= D, H, S or N
  VARIANT 57: note = X= A, H, M, E, G, S or T
  VARIANT 58: note = X= S, V, P, T, N or D
  VARIANT 59: note = X= I or L
  VARIANT 60: note = X= H, S, K, N, Y or E
  VARIANT 61: note = X= D or E
  VARIANT 62: note = X= T, I or A
  VARIANT 63: note = X= V or I
  VARIANT 64: note = X= E, T, Q, R or K
  VARIANT 66: note = X= L or V
  VARIANT 67: note = X= I, T or L
  VARIANT 68: note = X=I, M, F, Y or L
  VARIANT 72: note = X= N, T, R, D or S
  VARIANT 73: note = X= S, N, R, T or I
  VARIANT 75: note = X= S or N
  VARIANT 76: note = X= S or A
  VARIANT 77: note = X= N, I or K
  VARIANT 78: note = X= G, E or K
  VARIANT 79: note = X= N, Y, D or T
  VARIANT 80: note = X= V, K or I
  VARIANT 81: note = X= T, A or I
  VARIANT 83: note = X= S, L or T
  VARIANT 87: note = X= E or V
  VARIANT 93: note = X= E or K
  VARIANT 94: note = X= K or R
  VARIANT 95: note = X= N, T or S
  VARIANT 96: note = X= I or F
  VARIANT 97: note = X= K, N, A, or T
  VARIANT 101: note = X= Q, K or E
  VARIANT 104: note = X= V, I or K
  VARIANT 105: note = X= H or R
  VARIANT 112: note = X= N or Y
  VARIANT 113: note = X= T, S, P, L or A
  VARIANT 114: note = X= S or P
XWXXVXXDLXXIXXLXXXXHXDXTLYTXSXXHPXCKXTXMXCFLLELXVIXXEXXXXXXXXXXXNXXXLANXXLXXXXXXXEXGCKXCEELEXXXXXEFLXSFXXIVQMFIXXX

SEQ ID NO: 2 - protein, 114 aa, synthetic construct
  REGION 1..114: note = primate uinterleukin 15 consensus sequence
  VARIANT 1: note = X = N or I
  VARIANT 4: note = X = N or D
  VARIANT 10: note = X = K or R
  VARIANT 11: note = X = K or R
  VARIANT 13: note = X = E or Q
  VARIANT 16: note = X = I or T
  VARIANT 19: note = X = M or I
  VARIANT 30: note = X = D or G
  VARIANT 31: note = X = V or I
  VARIANT 34: note = X = S or R
  VARIANT 52: note = X = L or H
  VARIANT 55: note = X = G or N
  VARIANT 56: note = X = D or N
  VARIANT 57: note = X = A or T
  VARIANT 58: note = X = S, N or D
  VARIANT 60: note = X = H or N
  VARIANT 63: note = X = V or I
  VARIANT 67: note = X = I or L
  VARIANT 72: note = X = N or R
  VARIANT 73: note = X =S or I
  VARIANT 76: note = X =S or A
  VARIANT 79: note = X =N or T
  VARIANT 80: note = X =V or I
  VARIANT 93: note = X =E or K
  VARIANT 112: note = X = N or Y
  VARIANT 113: note = X = T or A
XWVXVISDLXXIXDLXQSXHIDATLYTESXXHPXCKVTAMKCFLLELQVISXESXXXXIXDTXENLXILANXXLSXNGXXTESGCKECEELEXKNIKEFLQSFVHIVQMFIXXS

SEQ ID NO: 3 - protein, 114 aa, Homo sapiens
  VARIANT 93: note = X= E or K
NWVNVISDLKKIEDLIQSMHIDATLYTESDVHPSCKVTAMKCFLLELQVISLESGDASIHDTVENLIILANNSLSSNGNVTESGCKECEELEXKNIKEFLQSFVHIVQMFINTS

SEQ ID NO: 4 - protein, 61 aa, synthetic construct
  REGION 1..61: note = mammalian sushi domain consensus sequence
  VARIANT 3: note = X= P or A
  VARIANT 5: note = X= M or V
  VARIANT 7: note = X= V or I
  VARIANT 13: note = X= W, R or Q
  VARIANT 16: note = X= S or N
  VARIANT 19: note = X= L or V
  VARIANT 20: note = X= Y, H or N
  VARIANT 26: note = X= I or V
  VARIANT 39: note = X= S or T
  VARIANT 41: note = X=T or I
  VARIANT 45: note = X=T or I
  VARIANT 45: note = X= L or I
  VARIANT 48: note = X= A or N
  VARIANT 51: note = X= V or A
  VARIANT 53: note = X= H or Y
CPXPXSXEHADIXVKXYSXXSRERYXCNSGFKRKAGTSXLXECVXNKXTNXAXWTTPSLKC

SEQ ID NO: 5 - protein, 64 aa, synthetic construct
  REGION 1..64: note = enlarged mammalian sushi domain consensus sequence
  VARIANT 1: note = X= T, V or I
  VARIANT 5: note = X= P or A
  VARIANT 7: note = X= M or V
  VARIANT 9: note = X= V or I
  VARIANT 15: note = X= W, R or Q
  VARIANT 18: note = X= S or N
  VARIANT 21: note = X=L or V
  VARIANT 22: note = X=Y, H or N
  VARIANT 28: note = X= I or V
  VARIANT 41: note = X= S or T
  VARIANT 43: note = X= T or I
  VARIANT 47: note = X= L or I
  VARIANT 50: note = X= A or N
  VARIANT 53: note = X= V or A
  VARIANT 55: note = X= H or Y
XTCPXPXSXEHADIXVKXYSXXSRERYXCNSGFKRKAGTSXLXECVXNKXTNXAXWTTPSLKCI

SEQ ID NO: 6 - protein, 61 aa, synthetic construct
  REGION 1..61: note = primate sushi domain consensus sequence
  VARIANT 3: note = X= P or A
  VARIANT 5: note = X= M or V
  VARIANT 13: note = X= W, R or Q
  VARIANT 20: note = X= Y or H
  VARIANT 26: note = X= I or V
  VARIANT 51: note = X= V or A
  VARIANT 53: note = X= V or A
CPXPXSVEHADIXVKSYSLXSRERYXCNSGFKRKAGTSSLTECVLNKATNXAXWTTPSLKC

SEQ ID NO: 7 - protein, 63 aa, synthetic construct
  REGION 1..63: note = enlarged primate sushi domain consensus sequence
  VARIANT 1: note = X= V or I
  VARIANT 5: note = X= P or A
  VARIANT 7: note = X= M or V
  VARIANT 15: note = X= W, R or Q
  VARIANT 22: note = X= Y or H
  VARIANT 28: note = X= I or V
  VARIANT 53: note = X=V or A
  VARIANT 55: note = X=H or Y
XTCPXPXSVEHADIXVKSYSLXSRERYXCNSGFKRKAGTSSLTECVLNKATNXAXWTTPSLKC

SEQ ID NO: 8 - protein, 61 aa, Homo sapiens
CPPPMSVEHADIWVKSYSLYSRERYICNSGFKRKAGTSSLTECVLNKATNVAHWTTPSLKC

SEQ ID NO: 9 - protein, 64 aa, Homo sapiens
ITCPPPMSVEHADIWVKSYSLYSRERYICNSGFKRKAGTSSLTECVLNKATNVAHWTTPSLKCI

SEQ ID NO: 10 - protein, 77 aa, synthetic construct
  REGION 1..77: note = mammalian sushi and domains consensus sequence
  VARIANT 1: note = X= T, V or I
  VARIANT 5: note = X= P or A
  VARIANT 7: note = X= M or V
  VARIANT 9: note = X= V or I
  VARIANT 15: note = X= W, R or Q
  VARIANT 18: note = X= S or N
  VARIANT 21: note = X= L or V
  VARIANT 22: note = X= Y, H or N
  VARIANT 28: note = X= I or V
  VARIANT 41: note = X= S or T
  VARIANT 43: note = X= T or I
  VARIANT 47: note = X= L or I
  VARIANT 50: note = X=A or N
  VARIANT 53: note = X= V or A
  VARIANT 55: note = X= H or Y
  VARIANT 55: note = X= H or Y
  VARIANT 68: note = X=A, S or L
  VARIANT 70: note = X= V, A, S or T
  VARIANT 71: note = X= H or R
  VARIANT 72: note = X= Q or H
  VARIANT 73: note = X= R, S or K
  VARIANT 75: note = X= A, V or P
  VARIANT 76: note = X= P or S
  VARIANT 77: note = X= P or T
XTCPXPXSXEHADIXVKXYSXXSRERYXCNSGFKRKAGTSXLXECVXNKXTNXAXWTTPSLKCIRDPXLXXXXPXXX

SEQ ID NO: 11 - protein, 77 aa, synthetic construct
  REGION 1..77: note = primate sushi and domains consensus sequence
  VARIANT 1: note = X= V or I
  VARIANT 5: note = X= P or A
  VARIANT 7: note = X= M or V
  VARIANT 15: note = X= W, R or Q
  VARIANT 22: note = X= Y or H
  VARIANT 28: note = X= I or V
  VARIANT 53: note = X= V or A
  VARIANT 55: note = X= H or Y
  VARIANT 68: note = X= A, S or L
  VARIANT 70: note = X= V, A or T
  VARIANT 71: note = X= H or R
  VARIANT 75: note = X= A or V
XTCPXPXSVEHADIXVKSYSLXSRERYXCNSGFKRKAGTSSLTECVLNKATNXAXWTTPSLKCIRDPXLXXQRPXPP

SEQ ID NO: 12 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 13 - protein, 20 aa, synthetic construct
  REGION 1..20: note = peptidic linker
SGGSGGGGSGGGSGGGGSLQ

SEQ ID NO: 14 - protein, 20 aa, synthetic construct
  REGION 1..20: note = peptidic linker
SGGSGGGGSGGGSGGGGSGG

SEQ ID NO: 15 - protein, 21 aa, synthetic construct
  REGION 1..21: note = peptidic linker
SGGGSGGGGSGGGGSGGGSLQ

SEQ ID NO: 16 - protein, 22 aa, synthetic construct
  REGION 1..22: note = peptidic linker
SGGGGSGGGGSGGGGSGGGGSG

SEQ ID NO: 17 - protein, 211 aa, synthetic construct
  REGION 1..211: note = RLI
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 234 aa, synthetic construct
  REGION 1..234: note = ANti-HER2Neu light chain
MARSVTLVFLVLVSLTGLYADIVMTQSHKFMSTSVGDRVSITCKASQDVNTAVAWYQQKPGHSPKLLIYSASFRYTGVPDRFTGNRSGTDFTFTISSVQAEDLAVYYCQQHYTTPPTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 464 aa, synthetic construct
  REGION 1..464: note = Anti-HER2Neu heavy chain
MARSVTLVFLVLVSLTGLYAQVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 600 aa, synthetic construct
  REGION 1..600: note = IL15-L22-anti HER2Neu heavy chain
MARSVTLVFLVLVSLTGLYAQVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - protein, 578 aa, synthetic construct
  REGION 1..578: note = IL15-anti HER2Neu Heavy chain
MARSVTLVFLVLVSLTGLYAQVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 22 - protein, 697 aa, synthetic construct
  REGION 1..697: note = ELI-L22-anti HER2Neu Heavy Chain
MARSVTLVFLVLVSLTGLYAQVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGGGSGGGGSGGGGSGGGGSGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 675 aa, synthetic construct
  REGION 1..675: note = RLI-anti HER2Neu Heavy chain
MARSVTLVFLVLVSLTGLYAQVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 427 aa, synthetic construct
  REGION 1..427: note = IL15-L22-ScFv fragment HER2Neu
MARSVTLVFLVLVSLTGLYADIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEEKNIKEFLQSFVHIVQMFINTS

SEQ ID NO: 25 - protein, 405 aa, synthetic construct
  REGION 1..405: note = IL15-ScFv fragment HER2Neu
MARSVTLVFLVLVSLTGLYADIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEEKNIKEFLQSFVHIVQMFINTS

SEQ ID NO: 26 - protein, 501 aa, synthetic construct [sequence omitted]

SEQ ID NO: 27 - protein, 479 aa, synthetic construct
  REGION 1..479: note = RLI-ScFv fragment HER2Neu frgament
MARSVTLVFLVLVSLTGLYADIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 22 aa, synthetic construct
  REGION 1..22: note = peptidic linker
AAGGGSGGGSGGGGSGGGGSAA

(19) United States
(12) Patent Application Publication
(10) Pub. No.: US 2024/0083963 A1
(43) Pub. Date: Mar. 14, 2024

(54) IL-15 AND IL-15R\ALPHA SUSHI DOMAIN BASED IMMUNOCYTOKINES

(71) Applicants: Cytune Pharma, Nantes (FR); INSERM (Institut National de la Santé et de la Recherche Médicale), Paris (FR)

(72) Inventors: Sebastien Daniel Morisseau, Donges (FR); Geraldine Teppaz, Nantes (FR); Yannick Laurent Joseph Jacques, Nantes (FR); Bruno Gilbert Marc Robert, Valflaunes (FR); Guy Luc Michel De Martynoff, Mont-St-Guibert (BE); David Béchard, Saint-Etienne de Montluc (FR)

(21) Appl. No.: 18/352,998

(22) Filed: Jul. 14, 2023

Related U.S. Application Data
(63) Continuation of application No. 17/068,628, filed on Oct. 12, 2020, now Pat. No. 11,753,454.
Application No. 17/068,628 is a division of application No. 16/012,174, filed on Jun. 19, 2018, now Pat. No. 10,899,816.
Application No. 16/012,174 is a continuation of application No. 15/214,032, filed on Jul. 19, 2016, now Pat. No. 10,626,155.
Application No. 15/214,032 is a continuation of application No. 14/129,188, filed on Jul. 3, 2014, now abandoned.

(30) Foreign Application Priority Data
Jun. 24, 2011 (EP) 11358005.4

(51) Int. Cl. C07K 14/54; A61K 38/20; C07K 14/715; C07K 16/32

(52) U.S. Cl. CPC C07K 14/5443; A61K 38/2086; C07K 14/7155; C07K 16/32; C07K 16/2887

(57) ABSTRACT

The present invention relates to an immunocytokine comprising (a) a conjugate, and (b) an antibody or a fragment thereof directly or indirectly linked by covalence to said conjugate, wherein said conjugate comprises (i) a polypeptide comprising the amino acid sequence of the interleukin 15 or derivatives thereof, and (ii) a polypeptide comprising the amino acid sequence of the sushi domain of the interleukin 15R alpha (IL-15Rα) or derivatives thereof; and uses thereof.

### PRIORITY CLAIM

This application is a continuation of U.S. patent application Ser. No. 17/068,628, filed Oct. 12, 2020, which is a division of U.S. patent application Ser. No. 16/012,174, filed Jun. 19, 2018, now U.S. Pat. No. 10,899,816, which is a continuation of U.S. patent application Ser. No. 15/214,032, filed Jul. 19, 2016, now U.S. Pat. No. 10,626,155, which is a continuation of U.S. patent application Ser. No. 14/129,188, filed Jul. 3, 2014, which claims priority to International Application No. PCT/EP2012/002650, filed Jun. 22, 2012, which claims the priority of European Patent Application No.11358005.4, filed Jun. 24, 2011, all of which are incorporated herein by reference in their entireties.

### REFERENCE TO SEQUENCE LISTING

This application contains a Sequence Listing in a computer readable form, submitted via EFS-Web. The entire contents of the text file entitled “MAI0005US5_Sequence_Listing.XML” created on Jul. 14, 2023, and having a size of 104,922 bytes, is incorporated herein by reference.

### FIELD OF THE INVENTION

The present invention relates to new immunocytokines and to their use as a medicine, in particular for the treatment of cancer.

### BACKGROUND

Immunotherapy, in medicine, refers to an array of treatment strategies based on the concept of modulating the immune system to achieve a prophylactic and/or therapeutic goal. In the past few years, immunotherapy has been used for the treatment or the prevention of several pathologies, particularly cancers. Since the development of the cell fusion technique for the production of monoclonal antibodies, a vast number of monoclonal antibodies have been produced by researchers. Then after, other techniques have been developed for the generation of monoclonal antibodies, including the B cell hybridoma technique and the EBV hybridoma technique to produce human monoclonal antibodies.

Monoclonal antibodies (Mab) can be developed to target almost any epitope. The property of specific recognition and binding to particular cells/molecules has encouraged the development of Mabs as diagnostic and therapeutic reagents for a variety of disease states. Recombinant DNA techniques have been used to produce chimeric or humanized antibodies to adapt their administration to humans. Currently, several monoclonal antibodies are commercialized and available for the treatment of cancers, infectious diseases, immune diseases etc. such as RITUXAN®, HERCEPTIN®, AVASTIN®.

Monoclonal antibodies are targeted molecules and able to localize within a specific zone (cells, tissues . . . ) such as a tumor tissues. This property has also led to the development of Mabs conjugated to various substances (payloads) in an effort to target specific molecules in the tumor sites called tumoral antigens. Such substances (payloads) can be toxins, drugs, radionuclides, prodrug compounds . . . Many of these linkages involve the chemical conjugation of the reactive moiety (payload) with a given preparation of antibody, a process which can be cumbersome and subject to variation (U.S. Pat. No. 4,671,958).

Among these new molecules, the immunocytokines are of particular interest. Said immunocytokines correspond to fusion proteins comprising an antibody and a cytokine. These proteins retain both antigen-binding capacity and cytokine activity.

The cytokines are a category of signalling proteins and glycoproteins that, like hormones and neurotransmitters, are used extensively in cellular communication. While hormones are secreted by specific organs into the blood, and neurotransmitters are related to neural activity, cytokines are a more diverse class of compounds in terms of origin and purpose. They are produced by a wide variety of hematopoietic and non-hematopoietic cell types and can have effects on both nearby cells or throughout the organism, sometimes strongly depending on the presence of other chemicals. The cytokine family consists mainly of smaller, water-soluble proteins and glycoproteins with a mass of between 8 and 30 kDa. Cytokines are critical to the functioning of both innate and adaptive immune responses. They are often secreted by immune cells which have encountered a pathogen as a way to activate and recruit more immune cells and increase the system's response to the pathogen. However, apart from their role in the development and functioning of the immune system, cytokines are also involved in several developmental processes during embryogenesis.

Among the cytokines, interleukin 15 (IL-15) is a cytokine with structural similarity to IL-2 that is secreted by mononuclear phagocytes (and some other cells) following infection by virus(es) or indirect stimulation by cells recognized as non-self or debilitated. This cytokine induces cell proliferation of natural killer cells; cells of the innate immune system whose main role is to kill virally infected cells. The protein encoded by this gene is a cytokine that regulates T and natural killer cell activation and proliferation.

The construction of immunocytokines on the basis of IL-15 would thus be of particular interest for the combination of the tumor-targeting assets of tumor-specific antibodies with the immunomodulatory effects of interleukin 15. Several immunocytokines using notably interleukin-2 (IL-2) have been already obtained and demonstrated very interesting and encouraging results in phase 2 oncology clinical trials. Some examples of these fusion proteins are described in several patent applications (U.S. Pat. No. 5,645,835, EP 0,305,967, WO 86/01533, EP 0,439,095, and WO 85/00974).

Thus, interleukin 15-based immunocytokine has been produced in HEK-293 cells and is disclosed in International patent application PCT WO 2007/128563 and in KASPAR et al. (Cancer Research, vol. 67(10), p:4940-4948, 2007).

Nevertheless, the inventors established that such interleukin 15-based immunocytokines have a very limited interleukin 15 activity, and that their production is very difficult notably in CHO cells with low yield and many protein contaminants.

Thus, there is still a need for interleukin 15-based immunocytokines that can be used in immunotherapies.

### SUMMARY OF THE INVENTION

The invention relates to an immunocytokine comprising: A) a conjugate, and B) an antibody or a fragment thereof directly or indirectly linked by covalence to said conjugate, wherein said conjugate comprises: (i) a polypeptide comprising the amino acid sequence of interleukin 15 or derivatives thereof, and (ii) a polypeptide comprising the amino acid sequence of the sushi domain of IL-15Rα or derivatives thereof

In a second aspect, the invention relates to a nucleic acid encoding for an immunocytokine as described above.

In a third aspect, the present invention provides a vector comprising a nucleic acid as described above.

In a fourth aspect, the present invention relates to a host cell genetically engineered with the polynucleotide or with the vector described previously. The present invention also relates to a method of producing a host cell genetically engineered expressing an immunocytokine according to the invention, said method comprising the steps of: (i) introducing in vitro or ex vivo a nucleic acid or a vector as described above into a host cell, (ii) culturing in vitro or ex vivo the recombinant host cell genetically engineered obtained and (iii), optionally, selecting the cells which express and/or secrete said immunocytokine.

In a preferred embodiment said host cell genetically engineered is an animal cell, and preferably a CHO cell.

In a fifth aspect, the present invention provides a pharmaceutical composition comprising the immunocytokine as described above, a nucleic acid encoding thereof, or a nucleic acid vector comprising said nucleic acid, eventually associated with a pharmaceutically acceptable carrier.

In a preferred embodiment, said composition comprises a further therapeutic agent, which is preferably an anticancer agent.

In a sixth aspect, the present invention relates to a pharmaceutical composition as described previously for treating cancer in a subject.

In seventh aspect, the present invention relates to the products containing: (i) an immunocytokine as describe above, a nucleic acid sequence coding therefore, or a vector comprising such a nucleic acid sequence, and (ii) a therapeutic agent, preferably an anticancer agent, as a combined preparation for simultaneous, separate, or sequential use for treating cancer in a subject.

In an eighth aspect, the present invention relates to a method for treating cancer in a subject comprising the step of administrating to said subject a pharmaceutical composition as described previously.

In a final aspect, the present invention relates to a method for treating cancer comprising the step of simultaneously, separately, or sequentially administrating to a subject in need thereof a therapeutically effective amount of: (i) an immunocytokine as describe above, a nucleic acid sequence coding therefore, or a vector comprising such a nucleic acid sequence, and (ii) a therapeutic agent, preferably an anticancer agent.

### BRIEF DESCRIPTION OF THE DRAWINGS

FIG. 1 shows the activity of IL15 anti-CD20 immunocytokines as compared to IL15.

FIG. 2 shows the activity of IL15 anti-GD2-O-acetylated immunocytokine as compared to IL15.

FIG. 3 shows the CD20, GD2-Oacetylated and HER-2 binding activity of IL15 anti-CD20, anti-GD2-O-acetylated and anti-HER2 IL-15 immunocytokines respectively.

FIG. 4 shows the IL-15Rα binding activity of IL15 anti-CD20 immunocytokine as compared to anti-CD20 antibody (Rituximab).

FIG. 5 shows the CD20, GD2-Oacetylated and HER-2 binding activity of IL15 anti-CD20, anti-GD2-O-acetylated and anti-HER2 RLI immunocytokines respectively.

FIG. 6 show the IL15Rα binding activity of RLI anti-CD20 and IL15 anti-GD2-O-acetylated immunocytokines.

FIG. 7 shows the activity of RLI anti-CD20 immunocytokines as compared to IL15.

FIG. 8 shows the activity of RLI anti-GD2-O-acetylated immunocytokines as compared to IL15.

FIG. 9 shows the anti-metastatic activity of anti-GD2-O acetylated immunocytokine as compared to anti-GD2-O acetylated antibody.

FIG. 10 shows the antitumor activity of anti-CD20 immunocytokine in Raji model.

FIG. 11 show the activity of IL-15 anti-HER2 immunocytokines as compared to IL15.

FIG. 12 shows the activity of RLI anti-HER2 immunocytokines as compared to IL15.

### DETAILED DESCRIPTION

The present invention is based on the discovery by the present inventors that, whereas the production of an immunocytokine comprising interleukin 15 leads to the loss of more than 90% of interleukin 15 activity, the production of RLI-based immunocytokines leads to innovative IL15 immunocytokines presenting a powerful biological activity on αβγ and βγ immune cells that is largely superior to IL-15-based immunocytokines.

Surprisingly, RLI-based immunocytokines with a full IgG monoclonal antibody present improved biological efficacy on βγ immune cells as compared to RLI alone or to scFv fragment antibody. This surprising gain of activity on βγ immune cells could be critical in terms of activation/reactivation of NK cells and T lymphocytes in the immunosuppressive environment.

Still surprisingly, and whereas an interleukin 15 immunocytokine necessitates the presence of a linker between the immunoglobulin and the interleukin 15 moieties so as to be active; the immunocytokine of the invention present a similar interleukin 15 activity with or without any linker between its respective immunoglobulin and cytokine parts. This unnecessary presence of a linker region could represent powerful arguments in terms of fusion protein immunogenicity, limiting the hinge regions generating novel antigenic epitope and immunogenicity and in terms of production yield with limited cleaved forms.

Still surprisingly, the immunocytokines of the invention are IL-15 superagonist showing an increased activity (-i.e. 10 to 100 fold) as compared to RLI alone.

Moreover, the inventors obtained a good yield of production of the immunocytokine of the invention in CHO cells, and this with a yield of more than 90%.

This is surprising since the production in the same cells of interleukin 15 immunocytokine in CHO cells was very difficult.

As immunocytokines have a limited serum half-life traditionally and as immunocytokines-related tumor localization rate is a critical issue to generate a robust antitumor effect, the specific biological activity of RLI-based immunocytokines permitting to activate immune cells at very low concentration represent an important innovative step in this field and could improve the efficacy of such biological compounds in cancer patients.

Finally, the strong activity of the immunocytokine of the invention enables to forecast a realistic therapeutic use for this immunocytokine, which should be administrated by injection at a dose of 2.5-1 mg/kg of subject or less, and even at a dose of 0.1 mg/kg or less. In fact, the low activity of interleukin 15 immunocytokines such as the one disclosed in International patent application WO 2007/128563 does not enable any realistic therapeutic use (i.e. obtaining a therapeutic effect required a dose of more than 20 μg immunocytokine with four daily injections in a mouse tumor model suggesting the need of a dose of more than 5 mg/kg immunocytokine for obtaining some therapeutic effect).

Consequently, one aspect the present invention relates to an immunocytokine comprising of: A) a conjugate, and B) an antibody or a fragment thereof directly or indirectly linked by covalence to said conjugate, wherein said conjugate comprises: (i) a polypeptide comprising the amino acid sequence of interleukin 15 or derivatives thereof, and (ii) a polypeptide comprising the amino acid sequence of the sushi domain of IL-15Rα or derivatives thereof.

The term “immunocytokine” refers to a molecule comprising an antibody or fragments thereof directly or indirectly linked by covalence to a cytokine or derivates thereof. Said antibody and said cytokine can be linked by a linker peptide.

### Conjugate of the Immunocytokine of the Invention

The term “interleukin 15” in its general meaning in the art and refers to a cytokine with structural similarity to IL-2 (GRABSTEIN et al., Science, vol. 264(5161), p:965-968, 1994). This cytokine is also known as IL-15, IL15 or MGC9721. This cytokine and IL-2 share many biological activities and they were found to bind common hematopoietin receptor subunits. Thus, they may compete for the same receptor, negatively regulating each other's activity. It has been established that IL-15 regulates T and natural killer cells activation and proliferation, and that the number of CD8+memory cells is shown to be controlled by a balance between this cytokine and IL2. IL-15 activity can be measured by determining its proliferation induction on kit225 cell line (HORI et al., Blood, vol. 70(4), p:1069-72, 1987), as disclosed in the Examples.

Said IL-15 or derivatives thereof have at least 10% of the activity of human interleukin-15 on the proliferation induction of kit225 cell line, preferably at least 25% and more preferably at least 50%.

Said interleukin 15 is a mammalian interleukin 15, preferably a primate interleukin 15, and more preferably a human interleukin 15.

Mammalian interleukin 15 can be simply identified by the skilled person. As an example, one can cite Interleukin 15 from Sus scrofa (Accession number ABF82250), from Rattus norvegicus (Accession number NP_037261), from Mus musculus (Accession number NP_032383), from Bos taurus (Accession number NP_776515), from Oryctolagus cuniculus (Accession number NP_001075685), from Ovies aries (Accession number NP_001009734), from Felis catus (Accession number NP_001009207), from Macaca fascicularis (Accession number BAA19149), from Homo sapiens (Accession number NP_000576), from Macaca mulatta (Accession number NP_001038196), from Cavia porcellus (Accession number NP_001166300), or from Chlorocebus sabaeus (Accession number ACI289).

As used herein, the term “mammalian interleukin 15” refers to the consensus sequence SEQ ID NO:1.

Primate interleukin 15 can be simply identified by the skilled person. As an example, one can cite Interleukin 15 from Sus scrofa (Accession number ABF82250), from Oryctolagus cuniculus (Accession number NP_001075685), from Macaca fascicularis (Accession number BAA19149), from Homo sapiens (Accession number NP_000576), from Macaca mulatta (Accession number NP_001038196), or from Chlorocebus sabaeus (Accession number ACI289).

As used herein, the term “primate interleukin 15” refers to the consensus sequence SEQ ID NO:2.

Human interleukin 15 can be simply identify by the skilled person and refers to the amino acids sequence SEQ ID NO:3.

As used herein, the term “interleukin 15 derivatives” refers to an amino acid sequence having a percentage of identity of at least 92.5% (i.e. corresponding to about 10 amino acids substitutions) with an amino acid sequence selected in the group consisting of SEQ ID NO:1, SEQ ID NO:2 and SEQ ID NO:3, preferably of at least 96% (i.e.

corresponding to about 5 amino acids substitutions), and more preferably of at least 98.5% (i.e. corresponding to about 2 amino acids substitutions) or of at least 99% i.e. corresponding to about 1 amino acid substitution). Such derivatives can be simply identified by the skilled person in view of its personal knowledge and of the teaching of the present patent application. As an example of such derivatives, one can cite those described in the International Patent Application PCT WO 2009/135031. It will also be understood that natural amino acids may be replaced by chemically modified amino acids. Typically, such chemically modified amino acids increase the polypeptide half-life.

As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developed by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar, Robert C., Nucleic Acids Research, vol. 32, p:1792, 2004). To get the best local alignment, one can preferably use the BLAST software with the BLOSUM 62 matrix. The identity percentage between two sequences of amino acids is determined by comparing these two sequences optimally aligned, the amino acids sequences being able to encompass additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

Preferably, the interleukin 15 derivatives are IL-15 agonist or superagonist. One skilled in the art can simply identified an IL-15-agonist or -superagonist. As an example of IL-15-agonist or -superagonist, one can cite the ones disclosed in the International patent application WO 2005/085282 or in ZHU et al. (J. Immunol., vol. 183(6), p:3598-607, 2009).

Still preferably, said IL-15 agonist or superagonist is selected in the group comprising/consisting of L45D, L45E, S51D, L52D, N72D, N72E, N72A, N72S, N72Y and N72P (in reference to sequence of human IL-15, SEQ ID NO:3).

As used herein the term “the sushi domain of IL-15Rα” has its general meaning in the art and refers to a domain beginning at the first cysteine residue (C1) after the signal peptide of IL-15Rα, and ending at the fourth cysteine residue (C4) after said signal peptide.

Said sushi domain corresponding to a portion of the extracellular region of IL-15Rα is necessary for its binding to IL-15 (WEI et al., J. Immunol., vol. 167(1), p:277-282, 2001).

Said sushi domain of IL-15Rα or derivatives thereof has at least 10% of the binding activity of the sushi domain of human IL-15Rα to human interleukin-15, preferably at least 25% and more preferably at least 50%. Said binding activity can be simply determined by the method disclosed in WEI et al. (abovementioned, 2001).

Said sushi domain of the IL-15Rα is the sushi domain of a mammalian IL-15Rα, preferably the sushi domain of a primate IL-15Rα and more preferably the sushi domain of the human IL-15Rα.

The sushi domain of a mammalian IL-15Rα can be simply identified by the skilled person. As an example, one can cite the sushi domain of a IL-15Rα from Rattus norvegicus (Accession number XP_002728555), from Mus musculus (Accession number EDL08026), from Bos taurus (Accession number) XP_002692113), from Oryctolagus cuniculus (Accession number XP_002723298), from Macaca fascicularis (Accession number ACI42785), from Macaca nemestrina (Accession number ACI42783), from Homo sapiens (Accession number CAI41081), from Macaca mulatta (Accession number NP_001166315), Pongo abelii (Accession number XP_002820541), Cercocebus torquatus (Accession number ACI42784), Callithrix jacchus (Accession number XP_002750073), or from Cavia porcellus (Accession number NP 001166314).

As used herein, the term “sushi domain of a mammalian IL-15Rα” refers to the consensus sequence SEQ ID NO:4.

Preferably, the polypeptide comprising the amino acid sequence of the sushi domain of a mammalian IL-15Rα refers to the consensus sequence SEQ ID NO:5.

The sushi domain of a primate IL-15Rα can be simply identified by the skilled person. As an example, one can cite sushi domains of IL-15Rα from Oryctolagus cuniculus, from Macaca fascicularis, from Macaca nemestrina, from Homo sapiens, from Macaca mulatta, Pongo abelii, Cercocebus torquatus, or Callithrix jacchus.

As used herein, the term “sushi domain of a primate IL-15Rα” refers to the consensus sequence SEQ ID NO:6.

Preferably, the polypeptide comprising the amino acid sequence of the sushi domain of a primate IL-15Rα refers to the consensus sequence SEQ ID NO:7.

The sushi domain of human IL-15Rα can be simply identified by the skilled person and refers to the amino acids sequence SEQ ID NO:8.

Preferably, the polypeptide comprising the amino acid sequence of the sushi domain of human IL-15Rα refers to SEQ ID NO:9.

As used herein, the term “derivatives of the sushi domain of the IL-15Rα” refers to an amino acid sequence having a percentage of identity of at least 92% (i.e. corresponding to about 5 amino acids substitutions) with an amino acid sequence selected in the group consisting of SEQ ID NO:4, SEQ ID NO:5, SEQ ID NO:6, SEQ ID NO:7, SEQ ID NO:8, and SEQ ID NO:9, preferably of at least 96% (i.e. corresponding to about 2 amino acids substitutions), and more preferably of at least 98% (i.e. corresponding to about 1 amino acids substitutions). Such derivatives comprise the four cysteine residues of the sushi domain of L-15Rα and can be simply identified by the skilled person in view of his/her general knowledge and of the teaching of the present patent application. It will also be understood that natural amino acids may be replaced by chemically modified amino acids. Typically, such chemically modified amino acids enable to increase the polypeptide half-life.

According to a preferred embodiment, the conjugate comprises (ii) a polypeptide comprising the amino acid sequence of the sushi and hinge domains of IL-15Rα or derivatives thereof.

The IL-15Rα hinge domain is defined as the amino acid sequence that begins at the first amino residue after the sushi domain and that ends at the last amino acid residue before the first potential site of glycosylation. In human IL-15Rα, the amino acid sequence of the hinge region consists of the fourteen amino acids which are located after the sushi domain of this IL-15Ralpha, in a C-terminal position relative to said sushi domain, i.e., said IL-15Ralpha hinge region begins at the first amino acid after said (C4) cysteine residue, and ends at the fourteenth amino acid (counting in the standard “from N-terminal to C-terminal” orientation).

Said sushi and hinge domains of IL-15Rα are the sushi and hinge domains of a mammalian IL-15Rα, preferably the sushi and hinge domains of a primate IL-15Rα and more preferably the sushi and hinge domains of the human IL-15Rα.

The amino acid sequence of the sushi and hinge domains of a mammalian IL-15Rα can be simply identified by the skilled person. As used herein, the term “sushi and hinge domains of a mammalian IL-15Rα” refers to the consensus sequence SEQ ID NO:10.

The amino acid sequence of the sushi and hinge domains of a primate IL-15Rα can be simply identified by the skilled person. As used herein, the term “sushi and hinge domains of a primate IL-15Rα” refers to the consensus sequence SEQ ID NO:11.

The amino acid sequence of the sushi and hinge domains of human IL-15Rα can be simply identified by the skilled person. As used herein, the term “sushi and hinge domains of human IL-15Rα” refers to the consensus sequence SEQ ID NO:12.

As used herein, the term “derivatives of the sushi and hinge domains of IL-15Rα” refers to an amino acid sequence having a percentage of identity of at least 93% (i.e. corresponding to about 5 amino acids substitutions) with an amino acid sequence selected in the group consisting of SEQ ID NO:10, SEQ ID NO:11, and SEQ ID NO:12, preferably of at least 97% (i.e. corresponding to about 2 amino acids substitutions), and more preferably of at least 98% (i.e. corresponding to about 1 amino acids substitution). Such derivatives comprise the four cysteine residues of the sushi domain of L-15Rα and can be simply identified by the skilled person in view of its general knowledge and of the teaching of the present patent application. It will also be understood that natural amino acids may be replaced by chemically modified amino acids. Typically, such chemically modified amino acids enable to increase the polypeptide half life.

Both polypeptides i) and ii) of the conjugate may be linked non-covalently such as in the complex disclosed in U.S. Pat. No. 8,124,084 B2. Said conjugate or complex can be simply obtained by providing a suitable amount of the polypeptide i), providing a suitable amount of the polypeptide ii), admixing both polypeptides under suitable pH and ionic conditions for a duration sufficient to allow complex (i.e. conjugate) formation, and optionally concentrating or purifying said complex. The polypeptides of the complex (i.e. conjugate) can be formed, for example, using a peptide synthesizer according to standard methods; by expressing each polypeptide separately in a cell or cell extract, then isolating and purifying the polypeptide. Optionally, the therapeutic polypeptide complex of the invention can be formed by expressing both polypeptides i) and ii) in the same cell or cell extract, then isolating and purifying the complexes, for example, using chromatographic techniques, such as affinity chromatography with antibodies to the lymphokine portion, the lymphokine receptor portion, or to the complex.

Both polypeptides i) and ii) of the conjugate may be also covalently linked using bifunctional protein coupling agents or in a fusion protein.

Bifunctional protein coupling agents are well known from the skilled person such as methods using them, and include, as examples, N-succinimidyl (2-pyridyldithio) propionate (SPDP), succinimidyl (N-maleimidomethyl) cyclohexane-l-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

The term “fusion protein” refers to a protein created through the joining of two or more genes which originally coded for separate proteins. It is also known as a chimeric protein. Translation of this fusion gene results in a single polypeptide with functional properties deriving from each of the original proteins. Recombinant fusion proteins are created artificially by recombinant DNA technology for use in biological research or therapeutics. A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

In a preferred embodiment, the conjugate is a fusion protein.

The amino acid sequence of interleukin 15 or derivatives thereof can be in a C-terminal or in an N-terminal position relative to the amino acid sequence of the sushi domain of IL-15Rα or derivatives thereof. Preferably, the amino acid sequence of the interleukin 15 or derivatives thereof is in a C-terminal position relative to the amino acid sequence of the sushi domain of IL-15Rα or derivatives thereof.

The amino acid sequence of interleukin 15 or derivatives thereof and the amino acid sequence of the sushi domain of IL-15Rα or derivatives thereof may be separated by a first “linker” amino acid sequence. Said first “linker” amino acid sequence may be of a length sufficient to ensure that the fusion protein form proper secondary and tertiary structures.

The length of the first linker amino acid sequence may vary without significantly affecting the biological activity of the fusion protein. Typically, the first linker amino acid sequence comprises at least one, but less than 30 amino acids e.g., a linker of 2-30 amino acids, preferably of 10-30 amino acids, more preferably of 15-30 amino acids, still more preferably of 15-25 amino acids, most preferably of 18-22 amino acids.

Preferred linker amino acid sequences are those which allow the conjugate to adopt a proper conformation (i.e., a conformation allowing a proper signal transducing activity through the IL-15Rbeta/gamma signaling pathway).

The most suitable first linker amino acid sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

Preferably, the first linker amino acid sequence comprises near neutral amino acids selected in the group comprising Gly (G), Asn (N), Ser (S), Thr (T), Ala (A), Leu (L), and Gln (Q), most preferably in the group comprising Gly (G), Asn (N), and Ser (S).

Examples of linker sequences are described in U.S. Pat. Nos. 5,073,627 and 5,108,910.

Illustrative flexible linkers that are more particularly suitable for the present invention include those coded by the sequences of SEQ ID NO: 13 (SGGSGGGGSGGGSGGGGSLQ), SEQ ID NO:14 (SGGSGGGGSGGGSGGGGSGG) or SEQ ID NO:15 (SGGGSGGGGSGGGGSGGGSLQ).

### Antibody of the Immunocytokine of the Invention

The term “antibody” refers to an immunoglobulin molecule corresponding to a tetramer comprising four polypeptide chains, two identical heavy (H) chains (about 50-70 kDa when full length) and two identical light (L) chains (about 25 kDa when full length) inter-connected by disulfide bonds. Light chains are classified as kappa and lambda. Heavy chains are classified as gamma, mu, alpha, delta, or epsilon, and define the antibody's isotype as IgG, IgM, IgA, IgD, and IgE, respectively. Each heavy chain is comprised of a N-term heavy chain variable region (abbreviated herein as HCVR) and a heavy chain constant region. The heavy chain constant region is comprised of three domains (CH1, CH2, and CH3) for IgG, IgD, and IgA; and 4 domains (CH1, CH2, CH3, and CH4) for IgM and IgE. Each light chain is comprised of a N-term light chain variable region (abbreviated herein as LCVR) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions. The functional ability of the antibody to bind a particular antigen depends on the variable regions of each light/heavy chain pair, and is largely determined by the CDRs.

The term “antibody”, as used herein, refers to a monoclonal antibody per se. A monoclonal antibody can be a human antibody, chimeric antibody and/or humanized antibody.

Advantageously, the term antibody refers to an IgG, such as IgG1, IgG2 (IgG2a or IgG2b), IgG3 and IgG4. Preferably, the term antibody refers to IgG1 or IgG2, and more preferably to IgG2a.

“Chimeric antibody” means an antibody that is composed of variables regions from a murine immunoglobulin and of constant regions of a human immunoglobulin. This alteration consists simply of substituting the constant region of a human antibody with the murine constant region, thus resulting in a human/murine chimera which may have sufficiently low immunogenicity to be acceptable for pharmaceutical use. A number of methods for producing such chimeric antibodies have yet been reported, thus forming part of the general knowledge of the skilled artisan (See, e.g., U.S. Pat. No. 5,225,539).

“Humanized antibody” means an antibody that is composed partially or fully of amino acid sequences derived from a human antibody germline by altering the sequence of an antibody having non-human complementarity determining regions (CDR). This humanization of the variable region of the antibody and eventually the CDR is made by techniques that are by now well known in the art. As an example, British Patent Application GB 2188638A and U.S. Pat. No. 5,585,089 disclose processes wherein recombinant antibodies are produced where the only portion of the antibody that is substituted is the complementarity determining region, or “CDR”. The CDR grafting technique has been used to generate antibodies which consist of murine CDRs, and human variable region framework and constant regions (See. E. g., RIECHMANN et al., Nature, vol. 332, p: 323-327, 1988). These antibodies retain the human constant regions that are necessary for Fc dependent effector function, but are much less likely to evoke an immune response against the antibody. As an example, the framework regions of the variable regions are substituted by the corresponding human framework regions leaving the non-human CDR substantially intact, or even replacing the CDR with sequences derived from a human genome (See e.g., Patent application US 2006/25885). Fully human antibodies are produced in genetically modified mice whose immune systems have been altered to correspond to human immune systems. As mentioned above, it is sufficient for use in the methods of the invention, to employ an immunologically specific fragment of the antibody, including fragments representing single chain forms.

A humanized antibody again refers to an antibody comprising a human framework, at least one CDR from a non-human antibody, and in which any constant region present is substantially identical to a human immunoglobulin constant region, i.e., at least about 85 or 90%, preferably at least 95% identical. Hence, all parts of a humanized antibody, except possibly the CDRs, are substantially identical to corresponding parts of one or more native human immunoglobulin sequences. For example, a humanized immunoglobulin would typically not encompass a chimeric mouse variable region/human constant region antibody. As an example, the design of humanized immunoglobulins may be carried out as follows:

when an amino acid falls under the following category, the framework amino acid of a human immunoglobulin to be used (acceptor immunoglobulin) is replaced by a framework amino acid from a CDR-providing non-human immunoglobulin (donor immunoglobulin): (a) the amino acid in the human framework region of the acceptor immunoglobulin is unusual for human immunoglobulin at that position, whereas the corresponding amino acid in the donor immunoglobulin is typical for human immunoglobulin at that position; (b) the position of the amino acid is immediately adjacent to one of the CDRs; or (c) any side chain atom of a framework amino acid is within about 5-6angstroms (center-to-center) of any atom of a CDR amino acid in a three dimensional immunoglobulin model (QUEEN et al., Proc. Natl. Acad. Sci. USA, vol. 88, p:2869, 1991). When each of the amino acids in the human framework region of the acceptor immunoglobulin and a corresponding amino acid in the donor immunoglobulin is unusual for human immunoglobulin at that position, such an amino acid is replaced by an amino acid typical for human immunoglobulin at that position.

The term “antibody fragment” as used herein refers to antibody fragment capable of reacting with the same antigen than its antibody counterpart. Such fragments can be simply identified by the skilled person and comprise, as an example, Fab fragment (e.g., by papain digestion), F_{ab}' fragment (e.g., by pepsin digestion and partial reduction), F(_{ab}')₂ (fragment (e.g., by pepsin digestion), F_{acb} (e.g., by plasmin digestion), F_{d} (e.g., by pepsin digestion, partial reduction and reaggregation), and also scFᵥ (single chain Fv; e.g., by molecular biology techniques) fragment are encompassed by the invention.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(_{ab}')₂ (heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

Preferably, said antibody fragment is a scFv fragment.

In a preferred embodiment, said antibody or fragment thereof is directed against an antigen related to tumor neovascularization or to tumor extracellular matrix, or against a tumoral antigen.

As used herein, an “antigen related to tumor neovascularization” refers to an antigen which is expressed by the neo-synthetized blood vessels present in the tumor.

As an example of such antigen, one can cite the EDA and the EDB domains of fibronectin, Endosalin/TEM1, Endoglin/105, PSMA or B7-H4.

As used herein, As used herein, a “antigen related to tumor extracellular matrix” refers to an antigen which is expressed in the extracellular matrix present in the tumor.

As an example of such antigen, one can cite the G45 fragment of laminin-332 (ROUSSELLE et al., Cancer Research, vol. 68(8), p:2885-94, 2008).

As used herein a “tumoral antigen” refers to an antigenic substance produced in tumor cells. Many tumoral antigen are well known from the skilled person and one can cite, as non limiting examples, CD-20, CEA, EGFR, GD2, EPCAM, MUC1, PSMA, CD-19, GD3, GM1, CAIX, GD2-O-acetylated or HER2.

CD-20 is a non-glycosylated phosphoprotein expressed during early pre-B cell development and remains until plasma cell differentiation. Specifically, the CD20 molecule may regulate a step in the activation process which is required for cell cycle initiation and differentiation and is usually expressed at very high levels on neoplastic (“tumor”) B cells. CD20, by definition, is present on both “normal” B cells as well as “malignant” B cells. Thus, the CD20 surface antigen has the potential of serving as a candidate for “targeting” of B cell lymphomas.

Concerning the antibodies directed against CD-20, one can cite rituximab (“RITUXAN®”) (U.S. Pat. No. 5,736,137); the yttrium-[90]-labeled 2B8 murine antibody designated “Y2B8” or “Ibritumomab Tiuxetan” ZEVALIN® (U.S. Pat. No. 5,736,137); murine IgG2a “BI,” also called “Tositumomab,” optionally labeled with ¹³¹I to generate the “¹³¹I-BI” antibody (iodine 131 tositumomab, BEXXAR®) (U.S. Pat. No. 5,595,721); and humanized 2H7; Ofatumumab, a fully humanized IgG1 against a novel epitope on CD20 huMax-CD20 (International patent application PCT WO 2004/035607). Among them, rituximab, ibritumomab tiuxetan, and tositumomab received market approval for the treatment of specific lymphoma, and Ofatumumab received market approval for the treatment of specific leukemia.

The CEA (carcinoembryonic antigen) glycoprotein is a tumor marker involved in cell adhesion.

Concerning the antibodies directed against CEA, one can cite arcitumomab (IMMUNOMEDICS).

The ErbB receptors are expressed in various tissues of epithelial, mesenchymal and neuronal origin. Under normal conditions, activation of the ErbB receptors is controlled by the spatial and temporal expression of their ligands, which are members of the EGF family of growth factors. Ligand binding to ErbB receptors induces the formation of receptor homo- and heterodimers and activation of the intrinsic kinase domain, resulting in phosphorylation on specific tyrosine kinase residues within the cytoplasmic tail. These phosphorylated residues serve as docking sites for various proteins, the recruitment of which leads to the activation of intracellular signaling pathways. Among ErbB receptors, EGFR and HER2 are known to play an essential role in regulating cell proliferation and differentiation. They have a strong tendency to assemble with other HER receptors into homo- and/or heterodimers upon extracellular growth factor binding, which results in various forms of signal transduction pathways activation, leading to either apoptosis, survival, or cell proliferation.

Concerning the antibodies directed against EGFR, one can cite the humanized monoclonal antibody 425, also designated as matuzumab (hMAb 425, U.S. Pat. No. 5,558,864; EP 0531 472), the chimeric monoclonal antibody 225 (cMAb 225), also designated as cetuximab (ERBITUX®; U.S. Pat. No. 7,060,808), and the fully human anti-EGFR antibody panitumumab (VECTIBIX®; U.S. Pat. No. 6,235,883). Among them, cetuximab and panitumumab were demonstrated to inhibit human colorectal tumors in vivo and both received marked approval.

Concerning the antibodies directed against Her2, one can cite the recombinant humanized version of the mouse antibody 4D5 (U.S. Pat. No. 5,677,171), designated as huMAb4D5-8, rhuMAb HER2, trastuzumab, or HERCEPTIN® (U.S. Pat. No. 5,821,337). This antibody received marketing approval in 1998 for the treatment of patients with metastatic breast cancer whose tumors overexpress the ErbB2 protein.

GD2 is a disialoganglioside expressed on tumors of neuroectoderma origin, including neuroblastoma and melanoma.

Concerning the antibodies directed against GD2, one can cite the murine IgG3 monoclonal antibody 3F8, which has been used in the treatment of neuroblastoma, or the murine IgG3 monoclonal antibody 8B6, which is specific of the 0-acetylated form of GD2 (International patent application PCT WO 2008/043777). Preferably, the antibody is directed against CD-20 (e.g. rituximab disclosed in U.S. Pat. No. 5,736,137), GD2-O-acetylated (e.g. the one disclosed in International patent application PCT WO 2008/043777) or HER2 (e.g. trastuzumab or HERCEPTIN® disclosed in U.S. Pat. No. 5,821,337).

Both conjugate and antibody or fragment thereof may be covalently linked using bifunctional protein coupling agents or in a fusion protein.

Bifunctional protein coupling agent methods are well known by the skilled person and have been previously disclosed. As an example, the skilled person can use the method disclosed in TILL et al. (Proc. Natl. Acad. U.S.A., vol. 86(6), p:1987-91, 1989)

In a preferred embodiment, the immunocytokine is a fusion protein.

In another preferred embodiment, the immunocytokine is a complex, preferably a complex comprising a conjugate between the polypeptides i) and ii), wherein the polypeptide i) or ii) is fused to an antibody or fragment thereof.

The polypeptide i), the polypeptide ii), or the conjugate can be in a C-terminal or in an N-terminal position relative to the amino acid sequence of the antibody or fragment thereof.

Preferably, the conjugate is a fusion protein and the amino acid sequence of the conjugate is in a C-terminal position relative to the amino acid sequence of the antibody or fragment thereof, most preferably in a C-terminal position relative to the amino acid sequence of at least one of the heavy chain constant region of the antibody or fragment thereof.

The amino acid sequence of the conjugate and the amino acid sequence of the antibody or fragment thereof may be separated or not by a second “linker” amino acid sequence.

In a particular embodiment, the immunocytokine of the invention is a fusion protein wherein the conjugate and the antibody or fragment thereof are not separated by any linker.

In fact, the inventors have surprisingly established that the immunocytokine of the invention does not necessitate any linker between the immunoglobulin and cytokine parts so as to be active.

As for the first linker amino acid sequence, said second “linker” amino acid sequence may be of a length sufficient to ensure that the fusion protein form proper secondary and tertiary structures.

The length of the first linker amino acid sequence may vary without significantly affecting the biological activity of the fusion protein. Typically, the first linker amino acid sequence comprises at least one, but less than 30 amino acids e.g., a linker of 2-30 amino acids, preferably of 10-30 amino acids, more preferably of 15-30 amino acids, most preferably of 15-25 amino acids.

As for the first linker amino acid sequence, the most suitable second linker amino acid sequences (1) will adopt a flexible extended conformation, (2) will not exhibit a propensity for developing ordered secondary structure which could interact with the functional domains of fusion proteins, and (3) will have minimal hydrophobic or charged characteristics which could promote interaction with the functional protein domains.

Preferably, the second linker amino acid sequence comprises near neutral amino acid selected in the group comprising Gly (G), Asn (N), Ser (S), Thr (T), Ala (A), Leu (L), and Gln (Q), most preferably in the group comprising Gly (G), Asn (N), and Ser (S).

As an example of a second linker amino acid sequence which is suitable for the present invention, one can cite the sequence SEQ ID NO:16 (SGGGGSGGGGSGGGGSGGGGSG) or SEQ ID NO:28 (AAGGGSGGGSGGGGSGGGGSAA).

### Nucleic Acids, Vectors and Recombinant Host Cells

In a second aspect the present invention relates to a nucleic acid encoding for a immunocytokine as described above, preferably an immunocytokine corresponding to a fusion protein.

Said nucleic acid corresponds to RNA or DNA, preferably to DNA. According to a preferred embodiment, the nucleic acid encoding the immunocytokine of the invention is operatively linked to a gene expression sequence, which directs the expression of the nucleic acid within a prokaryotic or an eukaryotic cell, preferably within an eukaryotic cell. The “gene expression sequence” is any regulatory nucleotide sequence, such as a promoter sequence or promoter-enhancer combination, which facilitates the efficient transcription and translation of the immunocytokine nucleic acid to which it is operatively linked. The gene expression sequence may, for example, be a mammalian or viral promoter, such as a constitutive or inducible promoter.

Constitutive mammalian promoters include, but are not limited to, the promoters for the following genes: hypoxanthine phosphoribosyl transferase (HPTR), adenosine deaminase, pyruvate kinase, beta-actin promoter, muscle creatine kinase promoter, human elongation factor promoter and other constitutive promoters. Exemplary viral promoters which function constitutively in eukaryotic cells include, for example, promoters from the simian virus (e.g., SV40), papilloma virus, adenovirus, human immunodeficiency virus (HIV), cytomegalovirus (CMV), Rous sarcoma virus (RSV), hepatitis B virus (HBV), the long terminal repeats (LTR) of Moloney leukemia virus and other retroviruses, and the thymidine kinase promoter of herpes simplex virus. Other constitutive promoters are known to those of ordinary skill in the art.

The promoters useful as gene expression sequences of the invention also include inducible promoters. Inducible promoters are expressed in the presence of an inducing agent. For example, the metallothionein promoter is induced to promote transcription and translation in the presence of certain metal ions. Others inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operationally joined nucleic acid. The gene expression sequences optionally include enhancer sequences or upstream activator sequences as desired. As used herein, the nucleic acid sequence encoding the immunocytokine of the invention and the gene expression sequence are said to be “operationally linked” when they are covalently linked in such a way as to place the expression or transcription and/or translation of the immunocytokine of the invention coding sequence under the influence or control of the gene expression sequence.

Two DNA sequences are said to be operationally linked if induction of a promoter in the 5′ gene expression sequence results in the transcription of the immunocytokine of the invention and if the nature of the linkage between the two DNA sequences does not (1) result in the introduction of a frame-shift mutation, (2) interfere with the ability of the promoter region to direct the transcription of the immunocytokine of the invention, or (3) interfere with the ability of the corresponding RNA transcript to be translated into a protein. Thus, a gene expression sequence would be operationally linked to a nucleic acid sequence coding for the immunocytokine of the invention if the gene expression sequence were capable of effecting transcription of that nucleic acid sequence such that the resulting transcript is translated into the desired polypeptide.

Advantageously, said nucleic acid sequence comprises an intron, since pre-mRNA molecules has often been demonstrated to improve production yields of recombinant molecules. Any sequences of intron may be sued, and as an example, one can cite tone ones disclosed in ZAGO et al. (Biotechnol. Appl. Biochem., vol. 52(Pt 3), p:191-8, 2009) and in CAMPOS-DA-PAZ et al. (Mol. Biotechnol., vol. 39(2), p:155-8, 2008).

The nucleic acid coding for the immunocytokine of the invention may be delivered in vivo alone or in association with a vector.

In a third aspect, the present invention relates to a vector comprising a nucleic acid as described above.

In its broadest sense, a “vector” is any vehicle capable of facilitating the transfer of the nucleic acid coding for the immunocytokine of the invention to the cells. Preferably, the vector transports the nucleic acid to cells with reduced degradation relative to the extent of degradation that would result in the absence of the vector. In general, the vectors useful in the invention include, but are not limited to, plasmids, cosmids, phagmids, episomes, artificial chromosomes, viruses, other vehicles derived from viral or bacterial sources that have been manipulated by the insertion or incorporation of the immunocytokine nucleic acid sequences.

Plasmid vectors are a preferred type of vector and have been extensively described in the art and are well known to those of skill in the art. See e.g., SANBROOK et al.,

“Molecular Cloning: A Laboratory Manual,” Second Edition, Cold Spring Harbor Laboratory Press, 1989. Not limiting examples of plasmids include pBR322, pUC18, pUC19, pRC/CMV, SV40, and pBlueScript, and other plasmids are well known to those of ordinary skill in the art. Additionally, plasmids may be custom designed using restriction enzymes and ligation reactions to remove and add specific fragments of DNA.

Preferably, the nucleic acid vector can include selectable markers that are active both in bacteria and in mammalian cells.

In a fourth aspect, the present invention relates to a host cell genetically engineered with the nucleic acid or with the vector described previously.

As used herein, the term “host cell genetically engineered” relates to host cells which have been transduced, transformed or transfected with the nucleic acid or with the vector described previously.

As representative examples of appropriate host cells, one can cite bacterial cells, such as E. coli, fungal cells such as yeast, insect cells such as Sf9, animal cells such as CHO or COS, plant cells, etc. The selection of an appropriate host is deemed to be within the scope of those skilled in the art from the teachings herein.

Preferably, the host cell genetically engineered is an animal cell, and most preferably CHO-S cell (INVITROGEN, cat N^{o} 11619-012).

Chinese hamster ovary (CHO) cells are frequently used in the biopharmaceutical industry for the manufacture of biologics such as recombinant proteins, antibodies, peptibodies, and receptor ligands. One of the reasons that CHO cells are often used is that these cells have an extensive safety track record for biologics production. This is considered to be a well-characterized cell line and, as a result, the safety testing required may be less rigorous in some respects (e.g., retroviral safety) than that required for other cell types. Nevertheless, the production of interleukin 15 is very difficult, especially in this cell.

Surprisingly, the inventors established that the immunocytokines of the invention are well produced in this cell, the obtained immunocytokines having further a very good purity and activity.

The introduction of the nucleic acid or of the vector described previously into the host cell can be done by methods well known from one of skill in the art such as calcium phosphate transfection, DEAE-Dextran mediated transfection, or electroporation.

The present invention also relates to a method of producing a host cell genetically engineered expressing an immunocytokine according to the invention, said method comprising the steps of: (i) introducing in vitro or ex vivo a nucleic acid or a vector as described above into a host cell, (ii) culturing in vitro or ex vivo the recombinant host cell genetically engineered obtained and (iii), optionally, selecting the cells which express and/or secrete said immunocytokine. Such recombinant host cells can be used for the production of immunocytokine of the invention.

### Pharmaceutical Composition Comprising the Immunocytokine of the Invention

A further object of the invention relates to a pharmaceutical composition comprising the immunocytokine as described above, a nucleic acid encoding thereof, or a vector comprising said nucleic acid, eventually associated with a pharmaceutically acceptable carrier.

The expression “pharmaceutically acceptable” refers to molecular entities and compositions that are physiologically tolerable and do not typically produce allergic or similar undesirable reactions, such as gastric upset, dizziness and the like when administered to a human. Preferably, as used herein, the expression “pharmaceutically acceptable” means approvable by a regulatory agency of the Federal or state government or listed in the U.S. Pharmacopeia or other generally recognized pharmacopeia for use in animals, and more particularly in humans.

The term “carrier” refers to a solvent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like.

The pharmaceutical composition comprises an “effective amount” of the immunocytokine of the invention, which effective amount is sufficient to inhibit the growth of cancer cells, preferably sufficient to induce the regression of tumor growth. The doses used for the administration can be adapted as a function of various parameters, in particular as a function of the mode of administration used, of the relevant pathology, or alternatively of the desired duration of treatment. Naturally, the form of the pharmaceutical composition, the route of administration, the dosage and the regimen naturally depend on the condition to be treated, the severity of the illness, the age, weight, and sex of the subject, etc. The ranges of effective doses provided below are not intended to limit the invention and represent preferred dose ranges. However, the preferred dose can be tailored to the individual subject, as is understood and determinable by one of skill in the art, without undue experimentation.

In view of the marked efficiency of the immunocytokine of the invention, the skilled person can plan to use very small doses for treating a subject. As a non limiting example, the immunocytokine of the invention can be can be administered by injection at a dose of 2.5mg/kg or 1 mg/kg of subject or less, preferably at a dose of 0.5 mg/kg or less or 0.25 mg/kg or less and most preferably at a dose of 0.1 mg/kg or less.

As an example, the pharmaceutical compositions of the invention can be formulated for topical, oral, intranasal, intraocular, intravenous, intramuscular or subcutaneous administrations and the like. Preferably, the pharmaceutical composition contains vehicles which are pharmaceutically acceptable for a formulation intended to be injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Suitable pharmaceutical carriers are described in “Remington's Pharmaceutical Sciences” by E. W. Martin.

The immunocytokine of the invention, nucleic acids coding therefore or nucleic acid vectors may be solubilized in a buffer or water or incorporated in emulsions, microemulsions, hydrogels (e.g. PLGA-PEG-PLGA triblock copolymers-based hydrogels), in microspheres, in nanospheres, in microparticles, in nanoparticles (e.g. poly(lactic-co-glycolic acid) microparticles (e.g. poly lactic acid (PLA) ; poly (lactide-co-glycolic acid) (PLGA); polyglutamate microspheres, nanospheres, microparticles or nanoparticles), in liposomes, or other galenic formulations. In all cases, the formulation must be sterile and fluid to the extent of acceptable syringability. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose.

Dispersions can also be prepared in glycerol, liquid polyethylene glycols, mixtures thereof and in oils. Under ordinary conditions of storage and use, these preparations contain a preservative to prevent the growth of microorganisms.

The immunocytokines according to the invention can be formulated into a composition in a neutral or salt form. Pharmaceutically acceptable salts include the acid addition salts (formed with the free amino groups of the protein) which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or a dispersion medium containing, for example, water, ethanol, polyol (for example, glycerol, propylene glycol, and liquid polyethylene glycol, and the like), suitable mixtures thereof, and vegetables oils. The immunocytokines of the invention may also be modified, by pegylation as an example, so as to increase its biodisponibility. When the immonocytokine of the invention has a nucleic acid form, the carrier can also be a vector, such as a virus (e.g., MVA, rAAV, lentivirus, etc.)

The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminium monostearate, gelatin, polyols, half-life enhancing covalent and non-covalent formulations.

There are numerous causes of peptide instability or degradation, including hydrolysis and denaturation. Hydrophobic interaction may cause clumping of molecules together (i.e., aggregation). Stabilizers may be added to reduce or prevent such problems.

Stabilizers include cyclodextrine and derivatives thereof (see U.S. Pat. No. 5,730,969). Suitable preservatives such as sucrose, mannitol, sorbitol, trehalose, dextran and glycerin can also be added to stabilize the final formulation. A stabilizer selected from ionic and non-ionic surfactants, D-glucose, D-galactose, D-xylose, D-galacturonic acid, trehalose, dextrans, hydroxyethyl starches, and mixtures thereof may be added to the formulation. Addition of alkali metal salt or magnesium chloride may stabilize a peptide. The peptide may also be stabilized by contacting it with a saccharide selected from the group consisting of dextran, chondroitin sulphuric acid, starch, glycogen, dextrin, and alginic acid salt. Other sugars that can be added include monosaccharides, disaccharides, sugar alcohols, and mixtures thereof (E.g., glucose, mannose, galactose, fructose, sucrose, maltose, lactose, mannitol, xylitol). Polyols may stabilize a peptide, and are water-miscible or water-soluble. Suitable polyols may be polyhydroxy alcohols, monosaccharides and disaccharides including mannitol, glycerol, ethylene glycol, propylene glycol, trimethyl glycol, vinyl pyrrolidone, glucose, fructose, arabinose, mannose, maltose, sucrose, and polymers thereof. Various excipients may also stabilize peptides, including serum albumin, amino acids, heparin, fatty acids and phospholipids, surfactants, metals, polyols, reducing agents, metal chelating agents, polyvinyl pyrrolidone, hydrolysed gelatin, and ammonium sulfate.

The promise of cytokine therapy does indeed derive from the identification of these novel cytokines but even more fundamentally, the field is greatly benefiting from the ever-expanding amount of preclinical data that convincingly demonstrate synergistic and/or novel biologic effects, which may be achieved through the use of several combinations of cytokines with complementary immune-stimulating capabilities. Potential therapeutic active agent combinations with RLI-based immunocytokines includes by example chemotherapeutic agents, antiangiogenic agents, or immunomodulatory agents.

In a preferred embodiment, the composition of the invention may comprise a further therapeutic active agent, such as chemotherapeutic agents, antiangiogenic agents, or immunomodulatory agents.

For chemotherapeutic agents, it has been demonstrated that their therapeutic effects could be mediated in part by an indirect effect on immune responses, either by inducing an immunogenic cell death, balancing the immunosuppressive environments, debulking the primary large tumor and then facilitating the immune attack or by inducing a transient lymphopenia followed by homeostatic lymphoproliferation. Many of them are well known from the skilled person and, and as an example of chemotherapeutic agent which can be combined with the immunocytokine of the invention, on can cite fludarabine, gemcitabine, capecitabine, methotrexate, taxol, taxotere, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A.

For antiangiogenic agents, it has been demonstrated that they have off-target effects on immune system and then could facilitate the tumor immune responses. As an example of antiangiogenic agent which can be combined with the immunocytokine of the invention, on can cite drugs targeting the vascular endothelial growth factor receptor (VEGFR) via its tyrosine kinase, such as sorafenib, sunitinib, and pazopanib, or the mammalian target of rapamycin (mTOR), such as temsirolimus and everolimus.

For immunomodulatory agents which can be combined with the immunocytokine of the invention, one can cite cytokines (IL-2, IL-7, IL-15, IL-12, IL18, IL-21, GM-CSF, G-CSF, IFNα, . . . ), chemokines/antiangiogenic cytokines (IP10, Mig, SDF-1, RANTES, . . . ), TLR agonists, and immunoregulatory antibodies (anti-CTLA4, anti-PD1, anti-TGFb, agonist anti-CD40, . . . ).

### Therapeutic Methods and Uses

In a further aspect, the present invention relates to a pharmaceutical composition as described previously for treating cancer in a subject, preferably of a pharmaceutical composition comprising an immunocytokine as described previously.

As used herein, the term “subject” denotes a mammal, such as a rodent, a feline, a canine or a primate, and most preferably a human.

In another aspect, the present invention relates to products containing: (i) an immunocytokine as describe above, a nucleic acid sequence coding therefore, or a vector comprising such a nucleic acid sequence, and (ii) a therapeutic agent, preferably an anticancer agent, as a combined preparation for simultaneous, separate, or sequential use for treating cancer in a subject.

In still another aspect, the present invention relates to a method for treating cancer in a subject comprising the step of administrating to said subject a pharmaceutical composition as described previously.

In a final aspect, the present invention relates to a method for treating cancer comprising the step of simultaneously, separately, or sequentially administrating to a subject in need thereof of a therapeutically effective amount of: (i) an immunocytokine as describe above, a nucleic acid sequence coding therefore, or a vector comprising such a nucleic acid sequence, and (ii) a therapeutic agent, preferably an anticancer agent.

In the context of the invention, the term “treating” or “treatment”, as used herein, means reversing, alleviating, inhibiting the progress of, or preventing the disorder or condition to which such term applies, or one or more symptoms of such disorder or condition. The term “treating cancer” as used herein means the inhibition of the growth of cancer cells. Preferably such treatment also leads to the regression of tumor growth, i.e., the decrease in size of a measurable tumor. Most preferably, such treatment leads to the complete regression of the tumor.

In the following, the invention is described in more detail with reference to amino acid sequences, nucleic acid sequences and examples. However, no limitation of the invention is intended by the details of the examples. Rather, the invention pertains to any embodiment which comprises details which are not explicitly mentioned in the examples herein, but which the skilled person finds without undue effort.

### EXAMPLES

### 1) Construction of Interleukin 15 Based Immunocytokines

### Construction of Anti-CD20 (Rituximab) and Anti-GD2-O-Acetylated Immunocytokines

The expression plasmids encoding for the anti-CD20 chimeric IgG light chains and anti-GD2-O-acetylated chimeric IgG light chains were kindly provided by Dr WATIER (Université François-Rabelais de Tours, France) and Dr BIRKLE (INSERM, Université de Nantes, U892, France) respectively. The chimeric IgG heavy chain sequences of each antibody were designed to be fused in 3′term with or without a linker of 22 amino-acid (SEQ ID NO:16) to IL15 (SEQ ID NO:3, wherein the amino acid at position 93 is K). These nucleotide sequences were synthesized and cloned in pcDNA3.1 plasmids by GENEART. The complete sequence of light and heavy chains of the anti-GD2-O-acetylated antibody (8B6) are disclosed in the patent application EP 2,076,542 Al and in CERATO et al. (Hybridoma, vol. 16(4), p:307-16, 1997). The complete sequence of light and heavy chains of the anti-CD20 antibody (2B8) are disclosed in the patent US 5,736,137 (ANDERSON et al. as the antibody called “C2B8”) and in REFF et al. (Blood, vol. 83(2), p:435-45, 1994).

### Plasmid DNA Preparation and Transfection Reagent

A 40kDa linear PEI was obtained from POLYSCIENCE. A 1 mg /mL stock solution was prepared by dissolving the PEI in water with heating, neutralizing by NaOH, and sterilizing by filtration through a 0.22 μm filter. The solution stock was aliquoted and stored at −20° C.

Plasmids DNA for transfections were purified using the plasmid purification kits following the manufacturer's protocol (MACHEREY-NAGEL) and sterilizing by filtration through a 0.22 μm filter.

### Production and Purification of the Immunocytokines

### 1-Transient Transfection in Suspension:

Routinely maintained CHO-S (INVITROGEN) cells were seeded at a density of 1×10⁶ cells/mL in POWERCHO™-2 Medium (LONZA) and cultured overnight at 37° C. in a shaking incubator (100 rpm) with 5% CO2. For transfection, cells were then diluted to 2×10⁶ cells/mL in CD-CHO medium (INVITROGEN). The transfection complexes were prepared in 10% of the culture volume using NaCl 150 mM. Expression constructs DNA (2.5 mg/L of culture volume, using a 1:2 ration of plasmid encoding heavy chain to plasmid encoding light chain) were mixed with PEI diluted in NaCl (10 mg/L of final culture volume) and incubated for 10 min at room temperature before adding to the culture. Cells were cultured in a shaking incubator (130 rpm) at 37° C. for 5 h before doubling the culture volume with PowerCHO2 medium. Supernatant were collected 5 days postransfection.

### 2-Stable Transfection on Adherent Cells

CHO-K1 cells (ATCC n° CCL-61) were grown in DMEM supplemented with 1-glutamine, 10% FCS and penicillin (100 units/ml)/streptomycin (100 μg/ml) and transfected with each vector using LIPOFECTAMINE™ 2000 reagent (INVITROGEN), as recommended by the manufacturer. Clones were selected by limit dilution with medium containing geneticin and hygromycin (0.5 mg/ml) or blasticin and hygromycin (5 μg/mL and 100 μg/mL) for the anti-GD2O-aceylated ICK and anti-CD20 ICK, respectively. Culture supernatant of each clone was assayed for bifunctional proteins production by ELISA. For the production of ICK, selected clones were amplified in 25% DMEM medium and 75% AIM™ medium (INVITROGEN). Cells were then maintained in 100% of AIM™, and supernatant were collected and replaced every 2 days, for 10 days.

### 3-Supernatant Purification:

Collected supernatant were centrifuged at 3000 rpm for 20 minutes at 4° C., equilibrated at pH 7.8 with NaOH and filtered through a 0.22 μm filter. The conditioned mediums were purified by affinity chromatography using a protein A column (GE) according to the manufacturer's instructions. The purified proteins were concentrated with a 50 kDa AMICON™ units (MILLIPORE). During this step, elution buffer was replaced by PBS. The Purified proteins were finally assayed by ELISA and absorbance measuring at 280 nm. Purity was evaluated by electrophoresis.

### 4-Detection of the Immunoglobulin Moiety by ELISA.

MAXISORP™ flat bottom microtiter plate (NUNC) was coated with 100 μL of goat anti-human antibody (UP892370, INTERCHIM) diluted in PBS to 1,5 μg/mL for h at 4° C. Plate was then blocked with 2004, of blocking buffer (1% BSA+0.1% TWEEN™ 20 in PBS) for 1 h at 37° C. Plate was then washed 3 times with washing buffer (0.1% TWEEN™ 20 in PBS) and sample diluted in blocking buffer were added and incubated 30 min at 37° C. (100 μL). After 3 washing, Peroxidase conjugated goat anti-human IgG1 (109-036-003, JACKSON) diluted 1:10000 was added and incubated for 30 min at 37° C. TMB substrate (INTERCHIM) was used to determine protein levels and plates were read at 450 nm. Purified Rituximab (ROCHE) was used to generate a standard curve on plate.

### 5-Detection of the Cytokine Moiety by ELISA.

MAXISORP™ flat bottom microtiter plate (NUNC) was coated with 100 μL of the anti-IL15 B-E29 (DIACLONE) diluted in carbonate buffer to 2 μg/mL for 16 h at 4° C. Plate was then blocked with 2004, of blocking buffer (1% BSA in PBS) for 1 h at 37° C. the plate was then washed 3 times with washing buffer (0.05% Tween™ 20 in PBS). Sample diluted in TBS+0,05% BSA were added and incubated 1 h 30 min at 37° C. (100 !IL). After 3 washing, biotinylated anti-IL15 antibody BAM 247 (R&D SYSTEM) diluted to 200 ng/mL was added and incubated for 1 h 30 min at 37° C. The plate was washed 3 times and peroxidase conjugated streptavidin was added dilution 1:1000. TMB substrate (INTERCHIM) was used to determine protein levels and plates were read at 450 nm. IL-15 (PEPROTECH) was used to generate a standard curve on plate.

The results have shown that the obtained preparation of immunocytokines comprises many protein contaminants (i.e., equal or superior to 25%). So as to reduce these protein contaminations, the two anti-GD2-O-acetylated/interleukin 15 immunocytokines have been subjected to another round of protein A SEPHAROSE™ purification.

After this second round of protein A SEPHAROSE™ purification, the purity of the ICK c8B6-122-IL15 and c8B6-IL15 was respectively of 70 and 90%.

### Proliferation Activity of the Immunocytokines

The interleukin-15 proliferation activity of the obtained immunocytokines was tested. The proliferative responses of Kit 225 and 32 Dβ cells to ICK were measured by [³H] thymidine incorporation. Cells were maintained in culture medium for 3 days, washed twice, and starved in medium without cytokine for 24 h or 4 h for Kit 225 and 32 DB, respectively. They were then seeded in multiwell plates at 10⁴ cells/well in 100 μl and cultured for 48 h in medium supplemented with increasing concentration of sample. Human rIL-15 and RLI were used as calibrator. Cells were pulsed for 16 h with 0.5 μCi/well of [³H] thymidine, harvested onto glass fiber filters, and cell-associated radioactivity was measured.

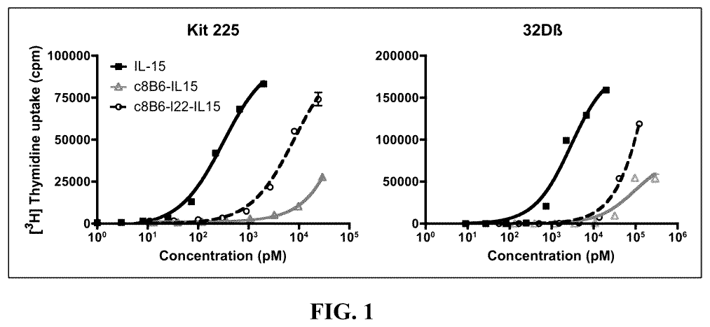

The FIG. 1 shows [³H]Thymidine incorporation by Kit 225 and 32 Dβ cells cultured with increasing concentrations of rIL-15 (▪), c8B6-IL15 (Δ), and c8B6-122-IL15 ( [symbol] ).

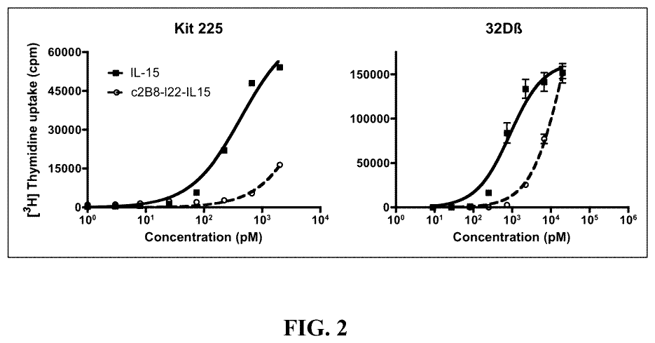

The FIG. 2 shows [³H]Thymidine incorporation by Kit 225 and 32 Dβ cells cultured with increasing concentrations of rIL-15 and c2B8-122-IL15 ( [symbol] ).

The results show that the biological activity of IL-15 is drastically decreased in the context of immunocytokine, meaning that conjugation of IL-15 with monoclonal antibody induces a loss of activity. Moreover, this loss is more important in absence of linker between the two moieties. It is to be noted that this loss of activity is more pronounced in the βγ context.

### Binding Activity of the Immunocytokines

The specific binding of the anti-CD20 and anti-GD2 0-acetylated ICK were was assessed by flow cytometry on tumors cells Raji and IMR32 respectively. The capacity of ICK to bind IL-15 receptor on effector cells were tested on Kit225. ICK coated on targeted cells were revealed with a PE-conjugated goat anti-human IgG mAb (PN IM0550, BECKMAN COULTER), or with a biotinylated mouse anti-IL15 antibody (BAM247, R&D SYSTEM) coupled to PE-streptavidin (SIGMA-ALDRICH). Targeted cells (1×10⁵) were incubated with each ICK for 1 h at 4° C., washed and then incubated with a PE-conjugate for 1 h at 4° C. Washed cells were finally analyzed on a FACSCALIBUR™ (BECTON DICKINSON).

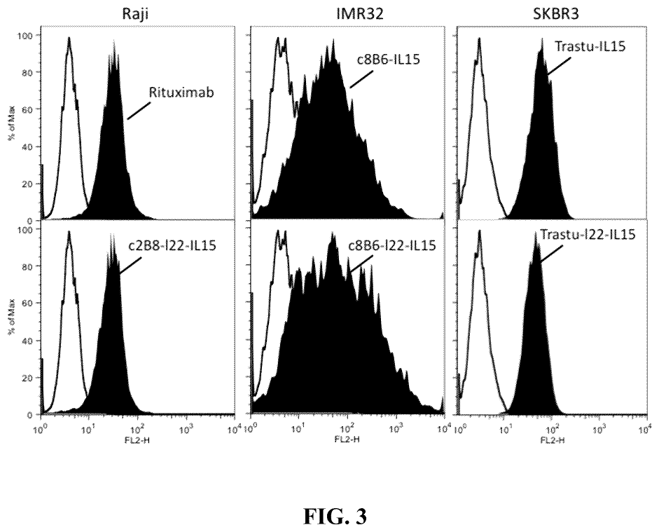

FIG. 3 shows flow cytometry evaluation of the ICK anti-CD20 (c2B8-122-IL15) and anti-GD2O-acetylated (c8B6-IL15 and c8B6-122-IL15) on CD20 expressing Raji cells and GD2O-acetylated expressing IMR32 cells. Cells were first incubated with ICK, then with a PE-conjugated goat anti-human IgG mAb for anti-CD20 or with biotinylated anti-IL15+PE-conjugated streptavidin for anti-CD20 and anti-GD2, respectively. Finally, sample were analysed on a FACSCALIBUR™. ICK were compared on Raji cells to the anti-CD20 Mab Rituximab (MABTHERA™, ROCHE).

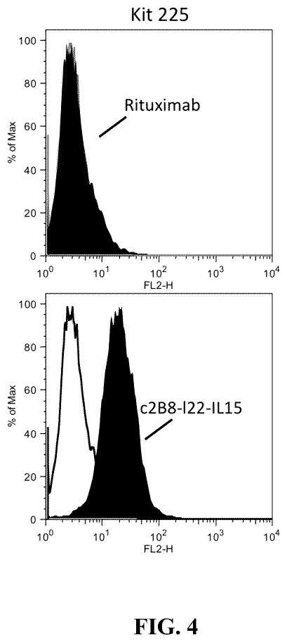

FIG. 4 shows flow cytometry evaluation of the ICK anti-CD20 (c2B8-122-IL15 and anti-GD2O-acetylated (c8B6-IL15 and c8B6-122-IL15) on IL15R expressing Kit 225 cells. Cells were first incubated with ICK, then with a PE-conjugated goat anti-human IgG mAb. Finally, sample were analysed on a FACSCALIBUR™. ICK were compared to the anti-CD20 Mab Rituximab (MABTHERA™, ROCHE).

The results show that the different immunocytokines bind to the IL-15 receptor and also to their respective tumor antigen target.

Thus, the loss of interleukin 15 activity in these immunocytokines is not the result of a loss of the binding of interleukin 15 on its specific receptor. Nevertheless, it appears that this existing binding does not permit to induce a normal cell proliferation

### Construction of RLI-Based Immunocytokines

### Construction of Anti-CD20 and Anti-GD2-O-Acetylated RLI Immunocytokines

The anti-CD20 and anti-GD2-O-acetylated immunocytokines were constructed as previously excepted that the IL15 Homo sapiens sequence was replaced by RLI2 sequence (SEQ ID NO:17).

### Production and Purification of the Immunocytokines

The production and purification of the immunocytokines were done as previously disclosed except that these immunocytokines were obtained with good yields and good purity (i.e. greater than 90%) after only one round of protein A SEPHAROSE™ purification.

### Binding Activity of the Immunocytokines

The specific binding of the anti-CD20 and anti-GD2 O-acetylated ICK RLI were was assessed by flow cytometry on tumors cells Raji, WM266.4 and IMR32. The capacity of ICK RLI to bind IL-15 receptor on effector cells were tested on Kit225. ICK RLI coated on targeted cells were revealed cells with a PE-conjugated goat anti-human IgG mAb (PN IM0550 BECKMAN COULTER), or with a biotinylated mouse anti-IL15 antibody (BAM247, R&D SYSTEM) coupled to PE-streptavidin (SIGMA-ALDRICH). Targeted cells (1×10⁵) were incubated with each ICK for 1 h at 4° C., washed and then incubated with a PE-conjugate for 1 h at 4° C. Washed cells were finally analyzed on a FACSCALIBUR™ (BECTON DICKINSON).

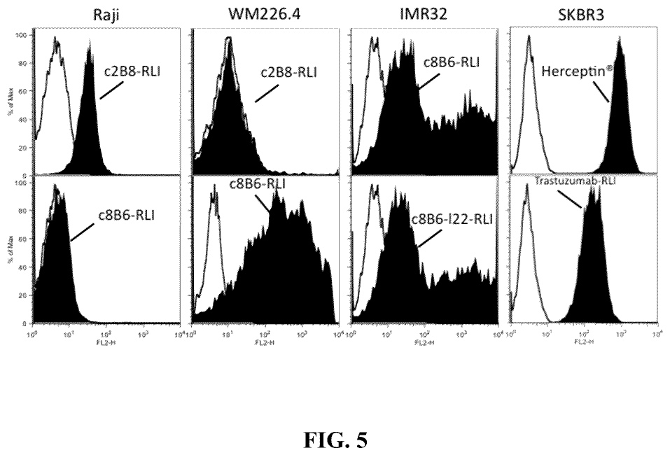

FIG. 5 shows flow cytometry evaluation of the ICK c2B8-RLI, c8B6-RLI and c8B6-122-RLI on CD20 expressing Raji cells and GD2O-acetylated expressing WM266.4 and IMR32 cells. Cells were first incubated with ICK RLI, then with a PE-conjugated goat anti-human IgG mAb for anti-CD20 or with biotinylated anti-IL15+PE-conjugated streptavidin for anti-CD20 and anti-GD2O-acetylated, respectively. Finally, samples were analysed on a FACSCALIBUR. ICK RLI were compared on Raji cells to the anti-CD20 Mab Rituximab (MABTHERA, ROCHE).

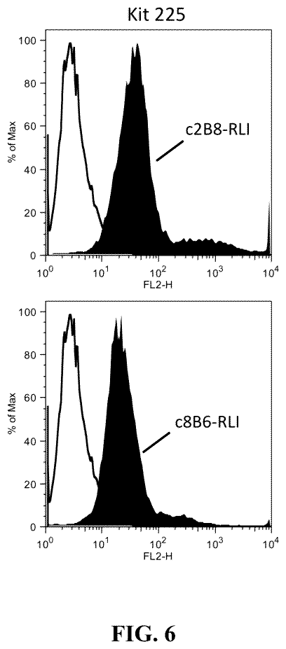

FIG. 6 shows flow cytometry evaluation of the ICK RLI anti-CD20 (c2B8-RLI) and anti-GD2O-acetylated (c8B6-RLI and c8B6-122-RLI) on IL15Rα expressing Kit 225 cells. Cells were first incubated with ICK RLI, then with a PE-conjugated goat anti-human IgG mAb. Finally, sample were analysed on a FACSCALIBUR™.

The results show that the immunocytokines of the invention bind to the IL-15 receptor and also to their respective tumor antigen target.

### Proliferation Activity of the Immunocytokines

The interleukin-15 proliferation activity of the newly obtained immunocytokines was tested.

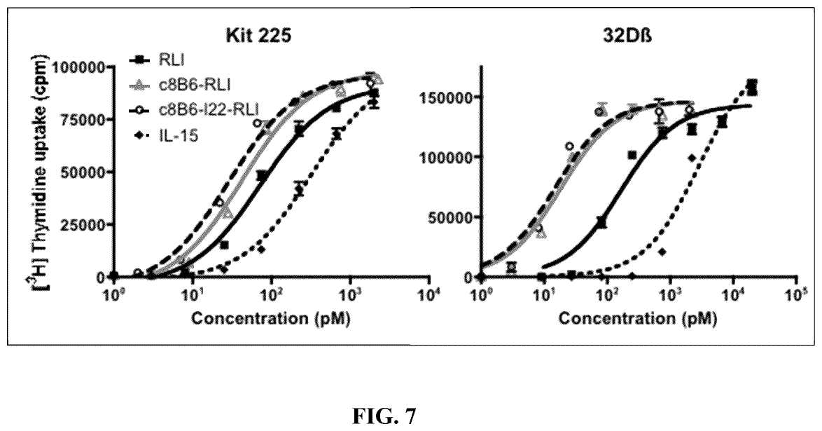

The FIG. 7 shows [³H]Thymidine incorporation by Kit 225 and 32 Dβ cells cultured with increasing concentrations of RLI (▪), rIL-15 (♦) c8B6-RLI (Δ), and c8B6-122-RLI ( [symbol] ).

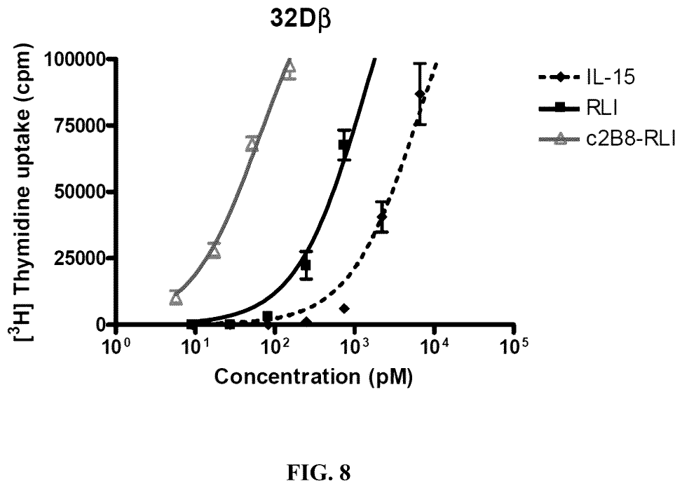

The FIG. 8 shows [³H]Thymidine incorporation by 32 Dβ cells cultured with increasing concentrations of RLI (▪), rIL-15 (♦) and c2B8-RLI (Δ).

The results show that the biological activity of IL-15 is conserved in the context of RLI-derived immunocytokines despite IL15 immunocytokines, meaning that conjugation of RLI with a monoclonal antibody permits surprisingly the conservation of this IL-15 activity. Moreover, this intriguing effect does not require any second linker between RLI and the monoclonal antibody. Surprisingly, it is to be noted that the RLI-derived immunocytokines present a significant gain of biological activity as compared to free IL-15 in the βγ context (about 10 to 100 fold increase).

### Antitumor Capability of the Anti-GD2-O-Acetylated Immunocytokine

The murine NXS2 neuroblatomas cell line was propagated in DMEM (10% FCS) under standard tissue culture conditions (37° C., 5% CO2). The NXS2 NB cell line expressing GD2-O-Ac was developed and characterized by LODE et al. (J. Natl. Cancer Inst., vol. 89(21), p:1586-94, 1997).

A/JOlaHsd mice, aged of 8 weeks, were purchased from HARLAN laboratories. Mice were housed at the animal facility of Inserm U892, which is approved by the French Association for Accreditation of Animal Care Laboratories and is maintained in accordance with the regulations and standards of Inserm Institute and the French Department of Agriculture.

Experimental hepatic metastases were induced by tail vein injection of 1×10⁵ NXS2 NB tumor cells in 200 μl of DMEM (pH 7.4). Treatment was initiated one day after tumor cell inoculation and consisted of 4 i.p. injections of 80 pmol of c8B6-RLI2 or c8B6 on day 1, 4, 7 and 11. Mice were sacrificed 25 days after graft and the hepatic tumor burden was evaluated by wet liver weight.

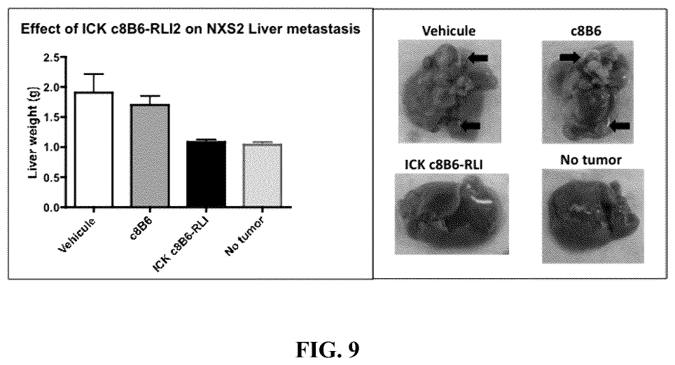

The FIG. 9 shows the efficacy of c8B6-RLI2 on NXS2 liver metastasis. C8B6 (12 μg) or c8B6-RLI (16 μg) was administered i.p. on days 1, 4, 7, and 11. Left: Graph represents mean of each group (n=5); bars, SEM. Right: representative pictures of liver, Arrows indicate some metastasis.

The results show that the mice which have received ICK remain liver metastasis free. Thus, and contrary to c8B6, ICK can eradicate the development of NXS2 liver metastasis, meaning that RLI conjugation to a monoclonal antibody dramatically enhances its antitumor capabilities.

### Antitumor Capability of the Anti-CD2O-RLI2 in Raji Model:

The human Raji B cells were cultured in RPMI1640 medium supplemented with 10% fetal calf serum, 2 mM 1-glutamine.

SCID CB-17 mice, aged 8 weeks, were purchased from the CHARLES RIVER Breeding Laboratories. Mice were kept under specific pathogen-free conditions in a separate facility using autoclaved cages of micro-isolator units and fed with irradiated solid food and sterilized water.

For inoculation, Raji cells were harvested in their log-phase, washed and re-suspended at 2.5×10⁶ cells/0.1 ml in phosphate-buffered saline (PBS) before being injected intravenously into the mice followed by ip treatment with immunocytokines 3 times a week (beginning on day 5) for 3 week after implantation. Mice received treatment in equimolar quantity except for the groups “immunocytokine” and “rituximab+RLI” which received a half dose. The mice were monitored daily for the presence of hind-leg paralysis and in that case sacrificed and scored as dead.

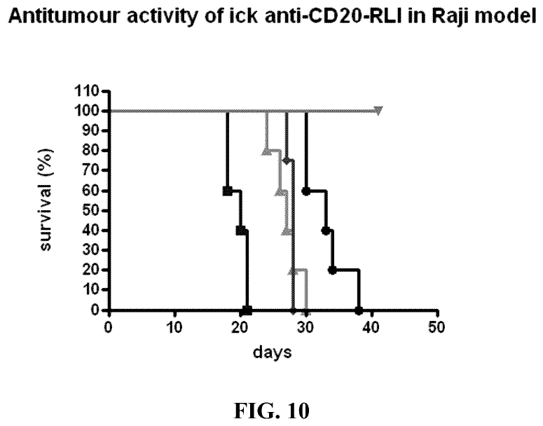

The FIG. 10 show the Kaplan Meier survival analysis of CB 17 SCID mice iv injected with Raji cells (n=5) and treated on J5-J7-J9; J12-J14-J16; J19-J21-J23 with PBS ( [symbol] ); RLI (▴; 2 μg) ; Rituximab (♦; 12 μg); Rituximab+RLI (●; 6 μg+1 μg), antiCD20-RLI (▾; 8 μg).

The results shows that the percentage of survival obtained in the Raji mice treated with RLI or with rituximab was similar and extend the 50% survival of tumour bearing mice from 20 to 27 days and to 28 days respectively relative to the PBS control.

The results show a further half increase of of the percentage of survival of tumour bearing mice for the “Rituximab+RLI” group, which increase is significantly different from the one obtained in the RLI group (P<0.01 or less).

Finally, and surprisingly, the results show that the treatment with anti-CD20 immunocytokine totally abrogates tumor development with no mouse death at the end of the experiment (day 50).

### Construction of Further Immunocytokines

### Construction of Anti-HER2Neu (Full IgG and ScFv) RLI and IL15 Immunocytokines

Sequence encoding for the anti-HER2 murine 4D5 IgG light chains, anti-HER2 murine IgG 4D5 heavy chains and anti-HER2 scFv were kindly provided by Dr DONDA (Biochemistry Institute Lausanne, Switzerland). The anti-HER2Neu IL15- and RLI-immunocytokines were constructed as previously on the basis of the anti HER2Neu light (SEQ ID NO:18) and heavy (SEQ ID NO:19) chains of the anti-HER2Neu antibody. For these constructions, sequence encoding the leader sequence of beta2 microglobulin in frame with sequence encoding chimeric IgG heavy chain sequences were designed to be fused in 3′term with or without a linker of 22 amino-acid (SEQ ID NO:16) to IL15 (SEQ ID NO:20 and 21 respectively) and to RLI (SEQ ID NO:22 and 23 respectively).

Constructions corresponding to sequence encoding the leader sequence of beta2 microglobulin in frame with sequence encoding anti-HER2Neu ScFv fragment fused in 3′term with or without a linker of 22 amino-acid to IL15 (SEQ ID NO:24 and 25 respectively) and to RLI (SEQ ID NO:26 and 27 respectively) were further designed and produced. These nucleotide sequences were synthesized by GENEART and sub-cloned in pCR3 (INVITROGEN) plasmids.

Biological and binding activities of these compounds are tested.

### Construction of Interleukin 15 Based Immunocytokines

### Plasmid DNA Preparation and Transfection Reagent

A 40 kDa linear PEI was obtained from POLYSCIENCE. A 1 mg /mL stock solution was prepared by dissolving the PEI in water with heating, neutralizing by NaOH, and sterilizing by filtration through a 0.22 μm filter. The solution stock was aliquoted and stored at −20° C.

Plasmids DNA for transfections were purified using the plasmid purification kits following the manufacturer's protocol (MACHEREY-NAGEL) and sterilizing by filtration through a 0.22 μm filter.

### Production and Purification of the Immunocytokines

### 1-Transient Transfection:

HEK293T cells, kindly provided by Dr. SCHNEIDER (Biochemistry Institute Lausanne, Switzerland) were seeded in T175 cm2 flask in DMEM-GLUTAMAX™ 10% SVF at 37° C. and 5% CO2. The day of transfection, a complex of DNA plasmid and PEI were prepared in sterile NaCl 150 mM. Plasmid DNA diluted in NaCl (1,25 mg/L of culture volume) were mixed with PEI diluted in NaCl (12,5 mg/L of culture volume) and incubated for 10 min at room temperature before adding to the cell culture. For anti-HER2 IgG-RLI or -IL15 immunocytokine a ratio of 1:2 DNA plasmid (heavy:light chain) were used. Cells were then cultured at 37° C. for 4 h. After this time medium was removed and fresh DMEN without SVF was added. Supernatant were collected 5 days postransfection.

### 2-Supernatant Purification:

Collected supernatant were centrifuged first at 1000 rpm for 5 minutes and secondly at 3000 rpm for 15 minutes at 4° C., adjusted to 20 mM sodium phosphate pH 8-9 as recommended by the manufacturer and filtered through a 0,22 μm filter. The conditioned medium was purified by affinity chromatography using a protein A column (GE) according to the manufacturer's instructions. The purified proteins were concentrated with a 50 kDa AMICON™ units (MILLIPORE) for IgG-ICK or 10 kDa for scFv-ICK. During this step, elution buffer was replaced by PBS. Proteins were finally assayed by ELISA and absorbance measuring at 280 nm. Purity was evaluated by electrophoresis.

### Binding Activity of the Immunocytokines

The specific binding of the anti-HER2 IgG-ICK or scFv-ICK was assessed by flow cytometry on HER2 positive cells SK-BR-3 using anti-IL15 antibody. The capacity of ICK to bind IL-15 receptor on effector cells were tested on Kit225. ICK coated on targeted cells were revealed with a FITC-conjugated goat anti-murine IgG mAb (SIGMA-ALDRICH) or with a FITC-conjugated mouse anti-IL15 antibody (R&D SYSTEM). Targeted cells (1×10⁵) were incubated with each ICK for 1 h at 4° C., washed and then incubated with a FITC-conjugate for 1 at 4° C. Washed cells were finally analyzed on a FACSCALIBUR™ (BECTON DICKINSON).

FIG. 3 shows flow cytometry evaluation of anti-HER2 (trastuzumab-IL15 and trastuzumab-122-IL15) on HER2 expressing SKBR3 cells.

FIG. 5 shows flow cytometry evaluation of the ICK Trastuzumab-RLI on HER2 expressing SKBR3 cells. Anti-HER2 ICK was compared on SKBR3 cells to the Trastuzumab (HERCEPTIN®, Genentech).

The results have shown the capacity of the ICK trastuzumab-RLI to coat tumor cell lines expressing the relevant TAA.

### Proliferation Activity of the Immunocytokines

The interleukin-15 proliferation activity of the fusion of IL-15 and Trastuzumab or anti-HER2 scFv fragments was tested on Kit 225 and 32 Dβ cells by measuring [³H]thymidine incorporation according to the method described previously.

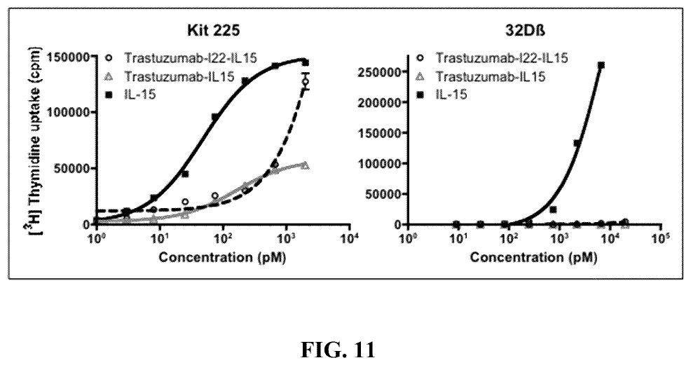

The FIG. 11 shows [³H]Thymidine incorporation by Kit 225 and 32 Dβ cells cultured with increasing concentrations of Trastuzumab-122-IL-15 ( [symbol] ), Trastuzumab-IL-15(Δ), and rIL-15 (▪).

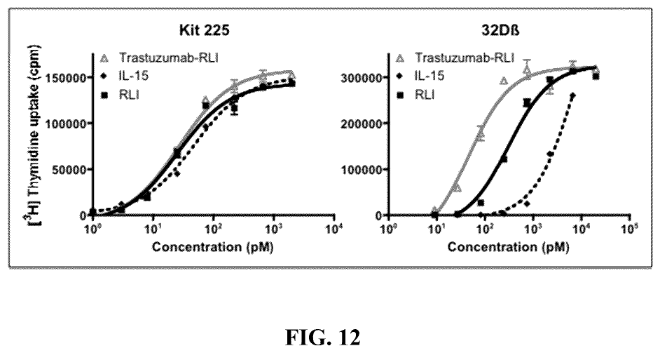

The FIG. 12 shows [³H]Thymidine incorporation by Kit 225 and 32 Dβ cells cultured with increasing concentrations of Trastuzumab-RLI (Δ), RLI (▪), and rIL-15 (♦).

The results show that the biological activity of IL-15 is drastically decreased on αβγ cells on the context of immunocytokine, meaning that conjugation of IL-15 with monoclonal antibody induces a loss of activity. Moreover, this loss is more important in absence of linker between the two moieties. On the βγ cells, conjugation completely abrogates the biological activity of IL-15 with or without linker.

In the context of RLI-derived immunocytokines despite IL15 immunocytokines, the results show that the biological activity of IL-15 is conserved meaning that conjugation of RLI with a monoclonal antibody permits surprisingly the conservation of this IL-15 activity. Moreover, this intriguing effect does not require any second linker between RLI and the monoclonal antibody.

Still surprisingly, the results show that the RLI-derived trastuzumab immunocytokines present a significant gain of biological activity as compared to free IL-15 in the βγ context (about 10 to 100 fold increase).

The results have further shown that, in the context of RLI-derived scFv immunocytokines despite IL15 immunocytokines, the biological activity of IL-15 is also conserved meaning that conjugation of RLI with a scFv fragment permits surprisingly the conservation of this IL-15 activity (data not shown). Again, this intriguing effect does not require any second linker between RLI and the scFv fragment (data not shown).

1-16. (canceled)

17. An immunocytokine comprising:
a) a conjugate, and
b) an antibody directly or indirectly linked by covalence to said conjugate,
wherein said conjugate comprises:
(i) a polypeptide comprising the amino acid sequence of the interleukin 15 or derivatives thereof having at least 92.5% sequence identity with the amino acid sequence of SEQ ID NO:3, and
(ii) a polypeptide comprising the amino acid sequence of the sushi domain of the IL-15Rα or derivatives thereof having at least 92% sequence identity with the amino acid sequence of SEQ ID NO: 8 or SEQ IDNO: 9,
wherein said antibody is directed against a tumor antigen or a tumor neovascularization antigen.

18. The immunocytokine of claim 1, wherein the polypeptides i) and ii) of the conjugate are covalently linked in a fusion protein.

19. The immunocytokine of claim 18, wherein the amino acid sequence of the conjugate and the amino acid sequence of the antibody are not separated by any linker amino acid sequence.

20. The immunocytokine of claim 18, wherein the amino acid sequence of the conjugate and the amino acid sequence of the antibody are separated by a second linker amino acid sequence.

21. The immunocytokine of claim 17, wherein said interleukin 15 has the amino acid sequence SEQ ID NO: 3.

22. The immunocytokine of claim 17, wherein sushi domain of IL-15Rα has the amino acid sequence SEQ ID NO: 8 or SEQ ID NO: 9.

23. The immunocytokine of claim 17, wherein the polypeptide (ii) comprising the amino acid sequence of the sushi domain of the 1L-15Rα or derivatives thereof has the amino acid sequence SEQ ID NO: 12.

24. The immunocytokine of claim 18, wherein said conjugate comprises the amino acid sequence of the interleukin 15 or derivatives thereof in a C-terminal position relative to the amino acid sequence of the sushi domain of the IL-15Rα or derivatives thereof.

25. The immunocytokine of claim 18, wherein the amino acid sequence of the interleukin 15 or derivatives thereof and the amino acid sequence of the sushi domain of the 1L-15Rα or derivatives are separated by a first linker amino acid sequence.

26. The immunocytokine of claim 17, wherein the antigen related to tumor neovascularization is selected from the group consisting of the EDA and the EDB domains of fibronectin, Endosalin/TEM1, Endoglin/105, PSMA and B7-H4.

27. The immunocytokine of claim 17, wherein the antigen related to the tumoral antigen is selected from the group consisting of CD-20, CEA, EGFR, GD2, EPCAM, MUC1, PSMA, CD-19, GD3, GM1, CAIX, GD2-O-acetylated and HER2.

28. The immunocytokine of claim 17, wherein the amino acid sequence of the conjugate is in a C-terminal position relative to the amino acid sequence of the antibody.

29. A pharmaceutical composition comprising the immunocytokine as defined in claim 17 eventually associated with a pharmaceutically acceptable carrier.

30. The pharmaceutical composition of claim 29, wherein said pharmaceutical composition is for treating cancer in a subject, preferably by an administration by injection at a dose of 2.5 mg/kg of subject or less.
